(19) United States
(12) Patent Application Publication
(10) Pub. No.: US 2026/0042822 A1
(43) Pub. Date: Feb. 12, 2026

(54) LYSOSOMAL DEGRADATION

(71) Applicant: Clear2Cure B.V., Rotterdam (NL)

(72) Inventors: Jord Cornelis STAM, Rotterdam (NL); Comelis Theodorus VERRIPS, Apeldoorn (NL); Paul Martinus Petrus VAN BERGEN EN HENEGOUWEN, Utrecht (NL)

(21) Appl. No.: 19/099,691

(22) PCT Filed: Jul. 28, 2023
(86) PCT No.: PCT/EP2023/071080

§ 371 Date: Jan. 29, 2025

(30) Foreign Application Priority Data
Jul. 29, 2022 (GB) 2211100.9

(51) Int. Cl. C07K 16/10; A61K 38/00; A61P 31/18; C07K 16/28; C07K 16/46

(52) U.S. Cl. CPC C07K 16/1063; A61K 38/00; A61P 31/18; C07K 16/1003; C07K 16/2863; C07K 16/468; C07K 2317/31; C07K 2317/569; C07K 2317/77; C07K 2319/00

(57) ABSTRACT

The present invention relates to bispecific antigen-binding polypeptides that may be used to remove unwanted agents, such as viruses or toxins, from the body and target them for degradation. The bispecific antigen-binding polypeptides of the invention have a first antigen-binding domain that binds an extracellular molecule and a second antigen-binding domain that binds to a cell surface protein, whereby the cell surface protein mediates internalisation of the bound complex. The invention also relates to pharmaceutical compositions comprising the bispecific antigen-binding poly peptides, and to methods of targeting an extracellular molecule for cellular internalisation and degradation via the lysosomal pathway.

### FIELD OF THE INVENTION

The present invention relates to bispecific antigen-binding polypeptides that may be used to remove unwanted agents, such as viruses or toxins, from the body and target them for degradation. The bispecific antigen-binding polypeptides of the invention have a first antigen-binding domain that binds an extracellular molecule and a second antigen-binding domain that binds to a cell surface protein, whereby the cell surface protein mediates internalisation of the bound complex. The invention also relates to pharmaceutical compositions comprising the bispecific antigen-binding polypeptides, and to methods of targeting an extracellular molecule for cellular internalisation and degradation via the lysosomal pathway.

### BACKGROUND OF THE INVENTION

The body encounters many unwanted agents, including infectious agents, viruses, microbial pathogens and also toxins, damaged proteins and other bodily products that have become toxic, including excess cytokines. To get rid of such unwanted, harmful agents, the body has evolved numerous mechanisms, many of which involve activation of the immune system. However, in pathogenic conditions, for instance HIV infection, the removal machinery of the body appears insufficient, most likely because the pathogenic agents have acquired evolutionary advantages such that they are capable of evading the body's natural clearing mechanisms.

Worldwide 6 out of 1000 people are infected with the human immunodeficiency virus (HIV). Over 25 million people have died since 1981 from acquired immunodeficiency syndrome (AIDS), the disease that is caused by HIV. Current antiretroviral cocktail medication have been effective in suppressing viral replication and reducing AIDS-related morbidity and mortality. However, antiretroviral therapy does not eliminate HIV that persists in a latent state, and it also cannot stop viral production from these latent reservoirs. Treatment interruption therefore results in renewed viral replication and a rapid viral rebound from these latent reservoirs (Kreider, E. F., & Bar, K. J. Current HIV/AIDS reports, 2022; doi.org/10.1007/s11904-022-00604-2). For this reason, lifelong daily adherence to antiretroviral therapy is required in infected individuals which is costly and thus not available to all infected individuals.

Broadly neutralizing antibodies (bNAbs) against HIV-1 envelope glycoproteins can be used as an alternative to antiretroviral therapy (Caskey, M. Current opinion in HIV and AIDS; 2020; 15:49-55). These bNAbs bind to one of several conserved epitopes present on the envelope glycoprotein gp120 (which has an essential role in the infection process), and can thereby inhibit viral entry into the target immune cells, and induce long-term suppression of viral replication. However, due to the high mutation rate of HIV, the major problem with using bNAbs is the emergence of antibody resistant viral variants. Moreover, upon binding of the bNAbs the viruses remain in the circulation and the rate of actual clearing of HIV viruses is not well defined and is most likely compromised in HIV patients.

Targeted protein degradation (TPD) is recognized as a promising new therapeutic modality designed to enhance clearance of unwanted agents in the body (Deshaies, R. J. Nature 2020; 580:329-338). Lysosome targeting chimeras (LYTACs) have been developed, which are heterobifunctional molecules comprising a recruiting molecule (e.g. an antibody) for a target protein joined by a linker to a ligand for a cell surface receptor, such as the cation-independent mannose-6-phosphate receptor (CI-M6PR) or the asialoglycoprotein receptor (ASGPR), which are cell-type specific lysosomal targeting receptors. For instance, a recruiting molecule can be tagged with a tri-GalNAc motif, a ligand which can engage the ASGPR for specific targeted degradation by hepatocytes (Ahn G, Banik S M, Miller C L, Riley N M, Cochran J R, Bertozzi C R. Nat Chem Biol. 2021; 17 (9): 937-946. In this way, extracellular proteins can be degraded by cells that specifically express these lysosomal targeting receptors.

A further approach is to use so-called ‘sweeping antibodies’. Sweeping antibodies require engineering of both the variable region and the constant region, for example as described in US patent publication U.S. Pat. No. 9,890,377B2. The variable region is engineered to enable the antibody to bind to an antigen in plasma and undergo pH-dependent dissociation from the antigen in the endosome, whereas the constant region is engineered to increase the cellular uptake of the antibody-antigen complex into the endosome via the FcRn receptor. This combination of modifications significantly accelerates the clearance of a soluble antigen from the circulation.

Both LYTACs and sweeping antibodies rely on exploiting cells in the body that have a natural function in clearing cargo. In general, the immune system has a remarkable adaptive efficacy to fight unwanted agents in the body. However, as mentioned above, in certain pathogenic conditions, for instance HIV infection, the virus has adapted to escape removal by these natural clearance mechanisms. Since the body's natural immune functions may be compromised, there remains a need to identify novel approaches for the rapid targeting of unwanted soluble agents for degradation that is independent of a functional immune system and does not rely on specialised cell types.

### SUMMARY OF INVENTION

The present inventors have developed a novel, alternative approach that is independent of a functional immune system which can be used to clear a broad range of unwanted soluble agents by capturing and redirecting them to non-specialised, non-immunological cells where they are internalised and degraded. The invention utilises bispecific antigen-binding molecules that are able to bind via one domain to the unwanted soluble agent in the circulation and via the other domain to cell surface proteins, such as membrane receptors, such that binding results in internalisation of the bispecific polypeptides including the bound unwanted agents. These cells are thus forced into becoming novel degradation units for the unwanted agents.

The inventors have shown that this novel approach can be used, for instance, to clear HIV by forced targeting of the virus to cells expressing epidermal growth factor receptor (EGFR), a receptor that is ubiquitously expressed in the body. By utilising a bispecific VHH antibody designed to bind to both HIV envelope proteins and EGFR, the inventors have shown that HIV can be efficiently recruited to EGFR (present on cells in the body to which the virus is not adapted), internalised and degraded in the lysosomal pathway.

In a first aspect, the present invention provides a bispecific antigen-binding polypeptide comprising:
a) a first antigen-binding domain that binds to an extracellular molecule; and
b) a second antigen-binding domain that binds to a cell surface protein.

In certain embodiments, the extracellular molecule is internalised and degraded when both the extracellular molecule and the cell surface protein are bound to the bispecific antigen-binding polypeptide.

In certain embodiments, the extracellular molecule is degraded via the lysosomal pathway.

In certain embodiments, the bispecific antigen-binding polypeptide is a bispecific antibody, a bispecific antibody fragment, or a bispecific VHH antibody. Preferably, the first antigen-binding domain is a VHH single domain and the second antigen-binding domain is a VHH single domain, optionally wherein the two VHH single domains are joined via a linker.

In certain embodiments, the extracellular molecule is a viral antigen, a toxin, a microbial pathogen, an allergen, a damaged or deregulated protein, an autoantibody, or other pathological or infectious agent. In certain preferred embodiments, the extracellular molecule is HIV envelope glycoprotein. In certain preferred embodiments, the extracellular molecule is a Sars-Cov2 spike protein.

In certain embodiments, the cell surface protein is epidermal growth factor receptor (EGFR), low density lipoprotein receptor (LDLR), transferrin receptor (TfR), hepatocyte growth factor receptor (cMet), MHC Class II, vascular endothelial growth factor receptor (VEGFR) or other growth factor receptor, CD20, CD40, CTLA-4, OX-40, 4-1-BB or ICOS.

In certain embodiments, the cell is a fibroblast cell, epithelial cell, endothelial cell, blood cell or platelet.

In certain preferred embodiments of the bispecific antigen-binding polypeptide, the first antigen binding domain binds to HIV and the second antigen-binding domain binds to EGFR. In certain preferred embodiments of the bispecific antigen-binding polypeptide, the first antigen binding domain binds to Sars-Cov2 and the second antigen-binding domain binds to EGFR.

In a second aspect, the present invention provides a pharmaceutical composition comprising a bispecific antigen-binding polypeptide as disclosed herein.

In a third aspect, the present invention provides a method of targeting an extracellular molecule for cellular internalisation and degradation, the method comprising administration of a bispecific antigen-binding polypeptide or a pharmaceutical composition as disclosed herein to a subject in need thereof.

In particularly preferred embodiments, there is provided a method of treating or preventing HIV, the method comprising administration of a bispecific antigen-binding polypeptide or a pharmaceutical composition as disclosed herein to a subject in need thereof. In particularly preferred embodiments, there is provided a method of treating or preventing Sars-Cov2, the method comprising administration of a bispecific antigen-binding polypeptide or a pharmaceutical composition as disclosed herein to a subject in need thereof.

In certain embodiments, the bispecific antigen-binding polypeptide is administered orally, sublingually, topically, intravenously, intramuscularly, intradermally, transderamally, intraperitoneally, subcutaneously, nasally, vaginally, rectally or by inhalation.

### BRIEF DESCRIPTION OF THE DRAWINGS

FIG. 1: Bispecific VHH llama antibodies target HIV to EGFR ectodomain. (a) The VHHs in bispecific constructs retain good binding properties. Wells were coated with antigen, binding measured by ELISA. n=2, representative experiment shown. (b) Bispecific VHHs mediate binding of HIV-1 antigens to EGFR ectodomain. Schematic set-up [with 1=immobilised EGFR in wells, 2-(heterospecific) bihead VHHs and 3=IR800 labeled HIV-1 protein] and IR800-labeled gp140 UG membrane protein specifically targeted to EGFR in wells. n>2, representative experiment shown. (c) Overview of relative efficacies of targeting by bispecific VHHs. gp140 is gp140 (UG37), Virus like particle (VLP) is VLP_{HIV}, except for Hep1E1 it is VLP_{HBV}. Shown are quantitative IR800 FLISA results and for overview an IR800 scan of a representative microtiter plate (dotted circles mark the wells) is shown. n=2. (d) Efficient targeting of low and high concentration HIV-1 to the coated EGFR ectodomain. Viruses were pre-incubated with 18 nM or 3 nM biheads for 1 hour and bound to EGFR coated ELISA plate for 2 hours. Bound HIV was detected by a p24 ELISA. Left graph: Viral concentration in the input was 10.1, 4.9, 3.9 and 9.0 ng/100 μl for clade A (92UG037), clade C (96ZM651), and clade B reference strains Bal and HXB2, respectively. Right graph: The same with 10 times concentrated HXB2. Clade B, n=2, mean±sd; Clade A and C, n=1, duplo shown.

FIG. 2: Binding, internalisation and degradation in EGFR expressing cells. (a) Kinetics of binding and internalisation. Labeled antigen (gp140 UG37) was pre-incubated for 30 minutes with bispecific VHHs and incubated with cells. At indicated times, cells were washed showing the total bound antigens (wells B) or stripped to remove antigens at the cell surface showing the internalised antigens (wells I). Bound (B) and internalised (I) antigens were scanned and quantified for IR800. Upper panel is a IR800 scan of some wells for illustration. Lower panels, graphs for quantification. The difference between bound and internalised represents the amount that is still exposed to the surface of the cells. All four display similar kinetics of net binding, whereas internalisation is more variable. n=2, representative experiment shown. (b) Immune fluorescence shows that envelope proteins gp140 and Virus Like Particles internalise similarly. Covalently green fluorescently labeled antigen (gp140, VLP_{HBV}) was pre-incubated with bispecific VHHs and added to cells for 0, 90 min and 18 hrs. Blue are nuclei, scale bar: 5 μm. n=2, representative experiment shown. (c) Bispecific VHH mediated internalisation results in rapid degradation of HIV proteins in the cells. Development of IR800 based internalisation and degradation assay. Top: Preincubated complexes IR800-labeled gp140 plus bispecific VHH H2E1 bound for 2 hours on ice to the indicated cells in 48-wells. After washing, chasing occurred in conditioned medium at 37° C. for the indicated time, in min. Cells were washed and scanned for IR800. Lower. The cells in the wells were next solubilized and proteins were separated and analyzed on a SDS-PAGE gel for IR800. Note that after binding (0 min chase) only full length HIV envelope protein is observed; after overnight chasing (o/n) mostly labeled protein degradation products are detected, in the front of the gel. n>2, representative experiment shown. (d) Comparison of the kinetics of degradation of IR800 labeled EGF, gp140 and VLPADW. After labeling gp140 and VLP_{HBV} were firstly preincubated with bispecific VHH H2E1 and Hep1E1, respectively. Next incubation was on Her14 cells on ice for 2 hours, cells were washed and complexes internalised for 0 min, 90 min, 180 min or overnight at 37° C. Subsequently cells were lysed, run on SDS-page gel and scanned for IR800. Next, the gel was blotted to PVDF to perform Western blot analyses with α-actin, to confirm equal loading. IR800 scan of the gels shows the IR800 labeled EGF, gp140 and VLPADW, resp. Intact protein and degraded protein are part of the same gel. Actin panel is a Western blot. n=2, representative experiment shown. graphs: IR800 quantification of intact proteins and total IR800 dye in the cells, n=2, mean±sd.

FIG. 3: Bispecific VHH mediated recruitment results in lysosomal degradation. (a) Colocalization of Alexa-488 fluorescently labeled EGF, gp140 and VLP (green) with lysotracker red in EGFR expressing cells. EGF was bound for 10 min and overnight, viral proteins were internalised overnight, mediated by bispecific VHHs H2E1 and Hep1E1 resp. Nuclei are blue, scale bar: 5 μm. n=2, representative experiment shown. (b) Chloroquine inhibits EGF degradation. SDS-PAGE gel of IR800 labelled EGF in cells. EGF was incubated on cells after 60 min pretreatment with chloroquine (μM indicated). Cells were lysed, run on SDS-PAGE gel and IR800 was detected. n=2, representative experiment shown. (c) Similarly, chloroquine inhibits gp140 and VLP_{HBV} degradation. SDS-PAGE gel of IR800 labelled gp140 and VLP_{HBV} internalised in cells after treatment with or without chloroquine (100 UM). Intact and degraded are in the same lane in the gel. n=2, representative experiment shown.

FIG. 4: Specific binding of infective HIV particles/virions to EGFR expressing cells results in internalisation and lysosomal degradation. (a) HIV bound to cells is internalised and degraded upon incubation. Pre-incubated HXB2 was incubated with Her14 and 14C cells (both expressing EGFR) for 3 hours (loading), and non-bound virus washed away. Viral protein was determined directly (loading) and after 21 hours, by measuring p24 in cell lysates by ELISA. n=2, mean±sd. (b) Trans-infectivity assay. Separately, after the indicated time points MT2 indicator cells were added to establish trans-infectivity of the bound HIV. Titre indicates the viral dilution at which syncytia are still formed. n=2, identical results. (c) Chloroquine inhibits degradation of internalised HIV. Her14 cells were treated (+) or not (−) with 100 μM chloroquine and incubated with virus for 3 hours, in the presence of heparin Then washed and incubated for 24 hours. HIV presence was determined by p24 ELISA. n=2, representative experiment shown. (d) The graph indicates the percentage of HIV that is not degraded after 24 hours as percentage of the input that is bound after 3 hours. n=2, mean±sd.

FIG. 5: Targeted degradation of SARS-COV-2 spike proteins by SARS-COV-2 specific bispecific single chain llama antibodies. (a) shows that the bispecific biheads C1-E3 and C2-E3 specifically bind and target the spike proteins to the EGFR expressing Her14 cells whereas the E3-E3 control bihead does not. A background amount of spike-IR800 protein binds also in the absence of biheads, yielding however a much lower signal. The PAGE gel analysis in (b) shows that the spike proteins targeted to the EGFR-expressing cells by C1-E3 and C2-E3 are largely degraded after 4 hours, similar to the EGF-IR800 control protein. For all of these the lower part of the gel shows small, labelled degradation fragments. The upper and lower panel in (b) derive from the same gel.

### DETAILED DESCRIPTION

### A. Definitions

Unless defined otherwise, all technical and scientific terms used herein have the same meaning as commonly understood by the ordinary person skilled in the art to which the invention pertains. Without limiting any term, further clarifications of some of the terms used herein are provided below.

As used herein, the term “Antibody” refers to polypeptides having a combination of two heavy and two light chains and which have significant known specific immunoreactive activity to an antigen of interest. There are five distinct classes of antibody that can be distinguished biochemically. All five classes of antibodies are within the scope of the present invention. The following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

The light chains of an antibody are classified as either kappa or lambda (κ,λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernible to the skilled artisan in view of the instant disclosure and, accordingly, are within the scope of the instant invention.

The variable region of an antibody allows the antibody to selectively recognize and specifically bind epitopes on antigens. That is, the VL domain and VH domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the VH and VL chains. The CDRs have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other.

As used herein, the term “VHH antibody” or “Heavy chain-only antibody” refers to a type of antibody produced only by species of the Camelidae family, which includes camels, llama, alpaca. These antibodies are composed of two heavy chains and are devoid of light chains. Each heavy chain has a variable domain at the N-terminus, and these variable domains are referred to as “VHH” domains in order to distinguish them from the variable domains of the heavy chains of the conventional heterotetrameric antibodies i.e. the VH domains, described above. Cartilaginous fishes also have heavy-chain only antibodies (IgNAR, ‘immunoglobulin new antigen receptor’), from which single-domain antibodies called V_{NAR} fragments can be obtained.

As used herein, the term “VHH single domain antibody” or “Nanobody” refers to the variable domains (i.e. the target recognition portion) of non-conventional heavy chain-only Camelid VHH antibodies. VHH are designated single-domain antibodies or nanobodies (registered trademark of Ablynx) in reference to their small size in the nanometer range. Their smaller size, superior properties, and ease of manufacturing while retaining the targeting specificity of the whole Ig molecule make them perfect tools for diagnosis and clinical applications. Notably, nanobodies do not usually display any of the solubility and aggregation problems typical of VH domains of conventional antibodies. A notable difference between the camelid VHH and the human VH domain is the length and orientation of the CDR3 loop. The longer CDR3 of a VHH enlarges the potential interaction surface with the target antigen, allowing nanobodies to bind to unique epitopes that are not accessible to conventional mAbs.

As used herein, the term “Bispecific antigen-binding polypeptide” may refer to any single binding molecule that has the dual function of specifically binding to at least two different epitopes. The term “specificity” refers to the ability of an antibody to specifically bind (e.g., immunoreact with) a given target antigen. An antibody molecule, or fragment thereof, may be monospecific and contain one or more antigen-binding domains which specifically bind a single epitope on a single target antigen. Alternatively, an antibody molecule, or fragment thereof, may be “multispecific” and contain two or more antigen-binding domains which specifically bind different epitopes either within the same antigen or located within different target antigens. For example, a bispecific antigen-binding polypeptide has two antigen-binding domains capable of recognising and binding two different target epitopes or antigens. In order to achieve multiple specificities, multispecific antibodies, or fragments thereof, are engineered to include different combinations of antigen-binding domains. Accordingly, the bispecific antigen-binding polypeptide of the invention may be a bispecific antibody having different VH-VL pairs for binding to two different target epitopes or antigens.

Alternatively, the bispecific antigen-binding polypeptide of the invention may be bispecific VHH antibody. This may be a bispecific VHH heavy chain-only antibody having different VHH domains present on each of the two heavy chains. In addition, a bispecific VHH antibody may refer to a bispecific VHH single-chain antibody, engineered by combining at least two different VHH domains having specificity for different epitopes or antigens. In certain preferred embodiments, a bispecific VHH antibody of the invention is comprised of two VHH domains joined by a flexible linker. A bispecific VHH antibody may also be referred to herein as a “bihead”, in the sense that a bihead contains two functional groups. This can be bispecific antibodies that recognize different epitopes. A bispecific antibody may recognize two different epitopes on the same molecule (biparatopic antibody) or epitopes on different molecules. The bispecific antibodies that we refer to herein generally bind with one head to an extracellular unwanted agent, and with the other head to the cell membrane molecule that mediates internalisation and degradation.

As used herein, the term “Antigen-binding domain” refers to a portion of an intact antibody that is responsible for selectively binding the target antigen. In particular, an antigen-binding domain includes polypeptide fragments of an immunoglobulin or antibody that bind the antigen or compete with intact antibody (i.e., with the intact antibody from which they were derived) for antigen binding. Exemplary antigen-binding domains include antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, a one-armed (monovalent) antibody, diabodies, triabodies, tetrabodies or any antigen binding molecule formed by combination, assembly or conjugation of such antigen binding fragments. The term antigen-binding domain as used herein is further intended to encompass antibody fragments selected from the group consisting of unibodies, domain antibodies and nanobodies (also termed VHH single domain antibodies). Preferably, the antigen-binding domain includes one or more CDRs that form all or part of the antigen-binding site. Fragments can be obtained, e.g., via chemical or enzymatic treatment of an intact or complete antibody or antibody chain or by recombinant means. An antigen binding agent in addition to an antibody can also comprise an aptamer or other entity which recognizes and binds the antigen.

### B. Format of the Bispecific Antigen-Binding Polypeptide

The present invention provides a bispecific antigen-binding polypeptide comprising: a) a first antigen-binding domain that binds to an extracellular molecule; and b) a second antigen-binding domain that binds to a cell surface protein. Upon binding of both the extracellular molecule and the cell surface protein to their respective antigen-binding domains, the complex is internalised into the cell and the extracellular molecule is subsequently targeted for degradation, preferably via the lysosomal pathway. Internalisation is mediated by the cell surface protein.

The bispecific antigen-binding polypeptide is not limited to any particular format, provided that it comprises a) a first antigen-binding domain that binds to an extracellular molecule; and b) a second antigen-binding domain that binds to a cell surface protein.

The bispecific antigen-binding polypeptide may be a bispecific antibody. For instance, the bispecific antibody may be a conventional heterotetrametic antibody having two variable domains that bind to different epitopes and/or different antigens. In other embodiments, the bispecific antigen-binding polypeptide may be a bispecific antibody fragment including but not limited to a F(ab′)2 fragment, tandem scFv, bi-specific T-cell engager (BiTE), diabody, triabody, tetrabody, or bi-nanobody.

The bispecific antigen-binding polypeptide may be a bispecific VHH antibody. In certain embodiments, the bispecific VHH antibody is a bispecific heavy chain-only antibody. Preferably, the bispecific VHH antibody is a single chain antibody comprised of at least two VHH single domains joined together. The bispecific VHH single chain antibody, also referred to herein as a “bihead” or “bi-nanobody”, may be engineered by combining two different VHH domains having specificity for two different epitopes or antigens. In certain preferred embodiments, the bispecific VHH antibody is comprised of two different VHH domains joined by a flexible linker. In certain preferred embodiments, the bispecific VHH antibody is comprised of two different VHH domains joined by a peptide linker. In certain preferred embodiments, the first antigen-binding domain is a VHH single domain and the second antigen-binding domain is a VHH single domain, optionally wherein the two VHH single domains are joined via a linker. For example, two different VHH domains may be combined via a GGGGGGGGS peptide linker sequence. However, the skilled person is aware of other suitable linkages that could be employed effectively to combine two VHH domains.

Bispecific molecules, including bispecific antibodies are known in the art and the skilled person knows how to produce such bispecific molecules. In brief, a first VHH domain (or nanobody) specific for a first target antigen (e.g. an extracellular molecule) may be generated or obtained by active immunization of a host species with a polypeptide comprising that antigen. A second VHH domain (or nanobody) specific for a second target antigen (e.g. a cell surface protein) may be generated or obtained by active immunization of a host species with a polypeptide comprising that antigen. For the production of VHH antibodies, any species from the family Camelidae, including llama species, may be immunized with a polypeptide including the respective antigen. By standard recombinant techniques the genes of the two VHHs are linked to each other via a linker sequence. Upon expression in cells, the bispecific VHH antibody (or bihead) proteins are purified. Production is possible in bacteria, yeast, eukaryotic expression cells etc.

Additional functional specificities can be added. For example, additional linkers and VHHs can be added to produce trihead antibodies with added specific properties. For instance, when injected into the blood the residence time of VHH antibodies in the circulation is less than that of conventional antibodies. One approach to increase the half-life of these molecules in vivo is to introduce a third VHH domain, for example an anti-albumin binding VHH domain, which will mediate binding to albumin, and prevent rapid clearing of the antibodies in vivo by the kidney. Accordingly, the bispecific antigen-binding polypeptides of the invention must comprise at least two antigen-binding domains but may, in certain embodiments, have more than two antigen-binding domains. In certain embodiments, the bispecific antigen-binding polypeptide further comprises (c) a third antigen-binding domain, optionally wherein the third antigen-binding domain binds to albumin.

The bispecific VHH antibody (bihead) format is particularly preferred due to the small size of the VHH domains. The biheads are composed of a combination of two VHH domains. VHHs are small single chain antibody fragments derived from llamas. VHH biheads have tremendous advantages: 1) they are easily constructed by cloning in bacteria; 2) they are easily produced and purified in high amounts, at low costs; 3) for treatment of humans they can readily be produced in yeast or other organisms that are acceptable for production of proteins to be used in clinical trials; 4) they are highly stable and resist harsh conditions and can be stored without refrigeration; and 5) they are non-immunogenic. Moreover, the VHH domains are particularly suitable for construction of bivalent, bispecific binding molecules, since multiple VHHs can easily be combined in one molecule.

Homology studies between camelid germline IgV gene repertoire and their human counterparts found 95% sequence identity of the camelid IGHV family 3 with its human FR counterpart. This means that nanobodies will have a low immunogenic profile and are thus suitable for human administration. Still, the sequence of a therapeutic nanobody can always be ‘humanized’ if desired.

### C. First Antigen-Binding Domain

As defined in the claims, the bispecific antigen-binding polypeptides comprise a first antigen-binding domain that binds to an extracellular molecule. The bispecific antigen-binding polypeptide may be engineered such that the first antigen-binding domain binds to any extracellular molecule of interest. In particular, it is an object of the present invention to bind to unwanted agents thus targeting these agents for removal from the circulation and subsequent degradation. In certain embodiments, the extracellular molecule is membrane-bound. In preferred embodiments, the extracellular molecule is soluble.

In certain embodiments, the extracellular molecule is a viral antigen, a toxin, microbial pathogen, an allergen, a damaged or deregulated protein, an autoantibody, or other pathological or infectious agent. In certain embodiments, the extracellular molecule is a viral antigen. In other words, the extracellular molecule is a viral antigen present on the surface of a virus.

In certain embodiments where the extracellular molecule is a viral antigen, the extracellular molecule is a viral antigen present on HIV, hepatitis, Sars-Cov2, influenza, herpes, Epstein Barr virus, adenovirus, flaviviruses, echovirus, rhinovirus, coxsackie virus, respiratory syncytial virus, pandemic Mumps virus, rotavirus, measles virus, rubella virus, parvovirus, vaccinia virus, HTLV virus, dengue virus, papillomavirus, molluscum virus, poliovirus, rabies virus, JC virus or arboviral encephalitis virus.

In other embodiments, the extracellular molecule is a damaged or deregulated protein that contributes to a disease state. In certain embodiments, the extracellular molecule is a cytokine or growth factor. In certain embodiments, the extracellular molecule is an interferon, an interleukin, a tumour necrosis factor (TNF), or a transforming growth factor b (TGFb). In specific embodiments, the extracellular molecule is type-1 interferon (IFN), IL-6, PDL-1, GM-CSF, Gal-3BP, BAG3, IL-17 family, EGF, VEGF, NRG1, NRG2, NRG3, NRG4, HGF, RANK ligand, TNF-α, soluble TNF-α receptor, IL-Ib, IL-5, IL-17 A, IL-12, IL-23, C5, BAFF, IgE or TGFb. In certain embodiments, the extracellular molecule is a protein that accumulates in a disease state, for example alpha-synuclein. In other embodiments, the extracellular molecule is a cholesterol carrier, such as ApoB or ApoE4. In other embodiments, the extracellular molecule is a clotting factor, such as Factor IX. In other embodiments, the extracellular molecule is a mucin, such as MUC1, MUC16, MUC2, MUC5AC, MUC4, CD43, CD45, or GPlb. In certain embodiments, the extracellular molecule is a hormone, such as insulin or ACTH.

In certain embodiments, the extracellular molecule is an autoantibody. Non-limiting examples of autoantibodies include: rheumatoid factor (RF), antinuclear antibody (ANA), Antineutrophil Cytoplasmic Antibodies (ANCA), Anti-Double Stranded DNA (anti-dsDNA), Anticentromere Antibodies (ACA), Antihistone Antibodies, Cyclic Citrullinated Peptide Antibodies (CCP), Extractable Nuclear Antigen Antibodies (e.g., anti-SS-A (Ro) and anti-SS-B (La), anti-RNP, anti-Jo-1, anti-Sm, Scl-70), Cardiolipin Antibodies, Beta-2 Glycoprotein 1 Antibodies, Antiphospholipid Antibodies (APA), Lupus anticoagulants (LA), Diabetes-related Autoantibodies, Anti-Tissue Transglutaminase (anti-tTG), Anti-Gliadin Antibodies (AGA), Intrinsic Factor Antibodies, Parietal Cell Antibodies, Thyroid Autoantibodies (e.g., anti-TPO, TSH receptor antibodies), Smooth Muscle Antibodies (SMA), Antimitochondrial Antibodies (AMA), Liver Kidney Microsome Type 1 Antibodies (anti-LKM-1), Anti-Glomerular Basement Membrane (GBM), or Acetylcholine Receptor (AChR) Antibodies.

In other embodiments where the extracellular molecule is a toxin, the extracellular molecule is toxic shock syndrome toxin (TSST-1), a snake toxin such as cobratoxin (Cbtx), a bacterial toxin such as clostridium toxin, or myeloperoxidase.

Antibodies, or antigen-binding domains, have also been raised against small toxins, proteinaceous and non-proteinaceous. These targets may also serve as targets of the present invention, since lysosomal targeting will result in inactivation of the toxin. Among such putative targets are opioids. Opioid-related fatal overdoses have reached epidemic proportions. Monoclonal antibodies are an emerging treatment strategy that targets and sequesters selected opioids in the bloodstream, reducing drug distribution across the blood-brain barrier, thus preventing or reversing opioid toxicity. Monoclonal antibodies were previously raised with high affinity and selectivity for oxycodone, morphine, fentanyl, and nicotine (Rodarte et al. 2023; Structure; 31 (1): 20-32). The present invention is particularly advantageous. The small size and uncomplicated nature of VHHs is beneficial for facilitating binding to these opioid targets. Moreover, the lysosomal-targeting method of the present invention will result not only in sequestering of the opioids, but also in rapid clearance. Accordingly, in certain embodiments, the extracellular molecule is an opioid. In certain embodiments, the extracellular molecule is selected from oxycodone, morphine, fentanyl, and nicotine.

In general, the extracellular molecule can be any agent to which first antigen-binding domains can bind. Particularly, the invention provides an alternative to known blocking antibodies. Whereas blocking antibodies specifically require the antigen-binding domain to bind to a functional, relevant epitope on the target, the bispecifics of the present invention can bind to any epitope on the target, provided that this results in rapid clearance. This is a substantial improvement over blocking antibodies which, once bound to their target, remain in the circulation for a substantial time increasing the risk of release of the toxic agent. Therefore, the high affinity binding requirements for blocking antibodies are much higher than the binding affinities required for the bispecifics of the present invention. Since binding of random epitopes on the target suffices, suitable antibodies are much more easily obtained and moreover several bispecifics binding to different epitopes can be used in combination, which will increase binding strength to the target.

The present invention also contemplates that the bispecific antigen-binding polypeptide may be used in conjunction with known therapies. For instance, the bispecific antigen-binding polypeptide may be used in conjunction with known blocking antibodies. In this way, the invention may be used to enhance the efficacy of already successful blocking antibody treatments in the clinic. As mentioned above, conventional blocking antibodies bind to their target but remain in the circulation for considerable time before they are cleared. Advantageously, the bispecifics of the present invention can be engineered such that the first antigen-binding domain binds to an already blocked antigen (i.e. binds to a blocking antibody-antigen complex), either by binding to a blocking antibody or by binding to an alternative epitope on the antigen. The inventors hypothesise that this approach could provide an additional boost since the bispecific would target the entire complex to a cell surface protein resulting in internalisation and degradation of the entire complex, including the targets. In this way the targets are not just blocked but rapidly cleared. Accordingly, in certain embodiments, the extracellular molecule is a blocking antibody-antigen complex. In certain embodiments, the first antigen-binding domain binds to a blocking antibody-antigen complex.

In certain embodiments, exemplified herein, the extracellular molecule is HIV. In other words, the extracellular molecule is a viral antigen present on HIV. In certain embodiments, the first antigen-binding domain binds to HIV. In certain embodiments, the first antigen-binding domain binds to a viral antigen present on HIV. In certain embodiments, the extracellular molecule is HIV-1. In certain embodiments, the first antigen-binding domain binds to HIV-1. The extracellular molecule may be present on any HIV-1 clade, and the first antigen-binding domain may be engineered to bind to any HIV-1 clade. In certain preferred embodiments, the extracellular molecule is an HIV envelope glycoprotein. In certain preferred embodiments, the first antigen-binding domain binds to an HIV envelope glycoprotein. In certain embodiments, the extracellular molecule is gp120, gp41 and/or gp140. In certain embodiments, the first antigen-binding domain binds to gp120, gp41 and/or gp140 on the surface of HIV.

In certain embodiments, a combination of different bispecific antigen-binding polypeptides may be produced wherein the first antigen-binding domain of each bispecific antigen-binding polypeptide is designed to bind to a different epitope of an HIV envelope glycoprotein. In this way, multiple bispecific antigen-binding polypeptides can be used to ensure effective targeting of a broad range of HIV subtypes (also called clades) and variants. The multiple binding also assures that single mutations that may limit binding of one of these bispecifics have a limited effect Thus, this approach can also be used to minimise the risk of escape mutants.

### D. Second Antigen-Binding Domain

As defined in the claims, the bispecific antigen-binding polypeptides comprise a second antigen-binding domain that binds to a cell surface protein. The bispecific antigen-binding polypeptide may be engineered such that the second antigen-binding domain binds to a specific cell surface protein thus directing the bound extracellular molecule to a chosen cell type. Alternatively, the bispecific antigen-binding polypeptide may be engineered such that the second antigen-binding domain binds to a ubiquitously expressed cell surface protein. Binding to the cell surface protein, for example a membrane receptor, will result in receptor-mediated endocytosis of the complex (i.e. the antigen-bispecific-receptor) and subsequent degradation of the internalised extracellular molecule.

Different cell types express a large variety of membrane proteins. Clearing functions have been assigned to some of these such that they act as “clearing receptors”. Typically, the presence of such clearing receptors on only certain cell types limits clearing ability to only those clearing receptor-expressing cells. In contrast, the present invention demonstrates that, irrespective of this attributed function, other membrane proteins can be exploited with the same result (i.e. clearing of the target) simply by binding to these membrane proteins, which has been shown to result in internalisation and degradation.

The cell surface protein may be any suitable cell surface protein capable of mediating endocytosis once bound by the bispecific antigen-binding polypeptide. Cellular receptors may be selected for their biological activity to induce internalisation of the complex into the cells, resulting in degradation of the unwanted extracellular molecule. Importantly, the cellular receptor does not have to be a natural receptor of the extracellular molecule. In certain preferred embodiments, the cell surface protein is not naturally involved in mediating lysosomal degradation of cargo. In certain embodiments, the cell surface protein is not a lysosomal targeting receptor. In certain embodiments, the cell surface protein is not a cation-independent mannose-6-phosphate receptor (CI-M6PR). In certain embodiments, the cell surface protein is not an asialoglycoprotein receptor (ASGPR). In certain preferred embodiments, the cell surface protein is not naturally involved in mediating lysosomal degradation of the extracellular molecule. In certain embodiments, the cell surface protein does not mediate lysosomal degradation of the extracellular molecule in the absence of the extracellular molecule and the cell surface protein being bound to the bispecific antigen-binding polypeptide. In certain embodiments, the second antigen-binding domain binds directly to the cell surface protein. In certain embodiments, the second antigen-binding domain binds indirectly to the cell surface protein, for example by binding to a ligand of the cell surface protein.

In certain embodiments, the cell surface protein is epidermal growth factor receptor (EGFR), low density lipoprotein receptor (LDLR), transferrin receptor (TfR), hepatocyte growth factor receptor (cMet), MHC Class II, vascular endothelial growth factor receptor (VEGFR) or other growth factor receptor, CD20, CD40, CTLA-4, OX-40, 4-1-BB or ICOS.

In certain embodiments, the cell surface protein is a member of the EGFR family, such as EGFR, ErbB2, ErbB3, or ErbB4. In certain embodiments, the second antigen-binding domain binds to EGFR, ErbB2, ErbB3, or ErbB4. In preferred embodiments, the cell surface protein is EGFR. In preferred embodiments, the second antigen-binding domain binds to EGFR. In preferred embodiments, the second antigen-binding domain binds to an epitope on the extracellular portion of EGFR.

The cell surface protein may be present on any desired cell type, provided that the cell is capable of lysosomal degradation of the extracellular molecule. A particular advantage of the present invention is that the unwanted extracellular molecule can be recruited to membrane proteins on cells that have not been described to be naturally involved in clearing cargo, but nevertheless are able to do so. The examples provided herein demonstrate that an unwanted extracellular molecule can be directed to EGFR, for instance, which is ubiquitously expressed in most tissues of the body. This forced targeting will result in cellular internalisation and degradation of the extracellular molecule. Without wishing to be bound by theory, it is hypothesised that the complex of the bispecific and bound extracellular molecule is piggybacking on the natural turnover of the EGFR. Interestingly, the inventors found that no EGF is required for this process. The membrane EGFR has many signalling functions. Most notably it rapidly reacts to binding to the growth factor EGF, resulting in activation of various signalling pathways. During this process the EGFR-EGF complex is internalised and targeted to the lysosome where both the receptor and the growth factor are degraded. The degradation of both the receptor and the growth factor is initiated by the binding of EGF to the EGFR. The present invention demonstrates that just binding by antibodies to the EGFR, even at sites that block EGF binding, still results in internalisation and degradation of the receptor and bound bihead, including degradation of the protein of interest that is bound to the bihead. This is a novel finding not anticipated from the function of the EGFR. It is different from biheads that recognise the EGFR with both heads, induce clustering of the EGFR in the membrane, and subsequently internalise and degrade the receptor.

In certain embodiments, the cell surface protein is present on a cell that is capable of lysosomal degradation of the extracellular molecule. In certain embodiments, the cell surface protein is present on a cell that does not naturally undergo lysosomal degradation of the extracellular molecule. In certain embodiments, the cell surface protein is present on a cell that does not undergo lysosomal degradation of the extracellular molecule in the absence of the extracellular molecule and the cell surface protein being bound to the bispecific antigen-binding polypeptide. In other words, although the cell may be capable of internalisation and degradation of the bound extracellular molecule, this event will not take place in the absence of the bispecific antigen-binding polypeptide. In certain embodiments, the cell surface protein is present on a cell that is (a) capable of lysosomal degradation of the extracellular molecule, and (b) does not undergo lysosomal degradation of the extracellular molecule in the absence of the extracellular molecule and the cell surface protein being bound to the bispecific antigen-binding polypeptide.

In certain embodiments, the cell is a non-immunological cell. In other words, the cell is not involved in the immune response. In certain embodiments, the cell is a fibroblast cell, an epithelial cell, endothelial cell, blood cell, or platelet. It may be beneficial if the cells are particularly abundant in the body. A surface of 1000 m² of endothelial cells is present in the blood vessels in the body. This provides a large capacity for degradation of unwanted agents that appear in the circulation. Therefore, in certain embodiments, the cell surface protein is present on endothelial cells. Alternatively, it may be advantageous to target cells that are present at certain locations. For many unwanted extracellular molecules, suitably localised cells can be employed that localise at the site of toxicity. In addition, local administration of the bispecific antigen-binding polypeptides is possible. For instance, epithelial cells that line the vaginal and anal walls can be targeted using microbicides that contain the bispecific antigen-binding polypeptides of the invention. This approach can be used for instance to capture HIV and reduce transmission following sexual intercourse. Therefore, in certain embodiments, the cell surface protein is present on epithelial cells.

It is possible that forced internalisation of an unwanted extracellular molecule, such as a virus, into cells might have the adverse effect that some infection occurs via this route. However, it is notable that not all cell types will provide the gene products required to support viral propagation. Accordingly, the present invention can be tailored depending on the pathogen to be removed. For instance, some cell types, such as platelets do not contain genetic material. This has the advantage that internalisation of infectious agents, such as viruses, by platelets can never result in unwanted replication of these agents. The invention thus allows targeting towards cells that will definitely not be able to support viral reproduction. Therefore, in certain embodiments, the cell surface protein is present on platelets. In other embodiments where the extracellular molecule is a viral antigen, the cell is not a natural target of the viral antigen. In other embodiments where the extracellular molecule is a viral antigen, the cell cannot support viral replication and/or propagation.

Degradation of the extracellular molecule preferably occurs via the lysosomal pathway. Thus, in certain preferred embodiments, the extracellular molecule is degraded via the lysosomal pathway. Lysosomes are ubiquitous organelles that can degrade proteins, nucleic acids, polysaccharides, and other biomaterials. Extracellular substances and cytoplasmic membrane proteins (such as receptors or channels) can enter the cell through endocytosis, traffic through the endosomal compartment, and undergo different steps of sorting, before they are either recycled to the plasma membrane, or delivered to the lysosome for degradation. In certain embodiments of the invention, the cell surface protein is recycled to the plasma membrane. In other embodiments of the invention, the cell surface protein is degraded along with the soluble agent.

### E. Targeting HIV for Degradation

As described in the below examples, the inventors have shown that the bispecific antigen-binding polypeptides of the invention can be used for the targeted removal and degradation of HIV. In this study, the inventors obtained proof of principle that the invention is suitable to target HIV for destruction to fibroblast cells that express the EGFR, as well as to epithelial derived (cancer) cells that express EGFR.

Accordingly, in certain embodiments, the bispecific antigen-binding polypeptide comprises:
a) a first antigen-binding domain that binds to HIV; and
b) a second antigen-binding domain that binds to a cell surface protein.

In certain embodiments, the bispecific antigen-binding polypeptide comprises:
a) a first antigen-binding domain that binds to HIV; and
b) a second antigen-binding domain that binds to EGFR.

In certain embodiments, the bispecific antigen-binding polypeptide comprises:
a) a first antigen-binding domain that binds to HIV; and
b) a second antigen-binding domain that binds to a cell surface protein on a non-immunological cell, a fibroblast cell, epithelial cell, endothelial cell, or platelet.

In certain embodiments, the bispecific antigen-binding polypeptide comprises:
a) a first antigen-binding domain that binds to HIV; and
b) a second antigen-binding domain that binds to EGFR on a non-immunological cell, a fibroblast cell, epithelial cell, endothelial cell, or platelet.

In each of the above embodiments, it is preferable that the extracellular molecule is internalised and degraded when both the extracellular molecule and the cell surface protein are bound to the bispecific antigen-binding polypeptide.

In each of the above embodiments, it is preferable that the extracellular molecule is degraded via the lysosomal pathway.

The present invention also provides a method of targeting HIV for cellular internalisation and degradation, the method comprising administration of a bispecific antigen-binding polypeptide or a pharmaceutical composition comprising same to a subject in need thereof.

The present invention further provides a method of treating or preventing HIV, the method comprising administration of a bispecific antigen-binding polypeptide or a pharmaceutical composition comprising same to a subject in need thereof.

The present invention further provides, a bispecific antigen-binding polypeptide for use in a method of treating or preventing HIV, the method comprising administration of a bispecific antigen-binding polypeptide or a pharmaceutical composition comprising same to a subject in need thereof.

The present invention further provides the use of a bispecific antigen-binding polypeptide for targeting HIV for cellular internalisation and degradation.

In each of the methods or uses described above, the bispecific antigen-binding polypeptide may be any of the bispecific antigen-binding polypeptides disclosed herein that bind to HIV. In particularly preferred embodiments of the methods, the bispecific antigen-binding polypeptide comprises a) a first antigen-binding domain that binds to HIV; and b) a second antigen-binding domain that binds to EGFR.

In each of the above embodiments in relation to targeting HIV for degradation, the skilled person is aware that the first antigen-binding domain may be engineered to bind to any epitope present on the surface of HIV. For instance, in certain embodiments, the first antigen-binding domain may bind to gp120, gp41 and/or gp140 on the surface of HIV. Similarly, the skilled person is aware that the second antigen-binding domain may be engineered to bind to other cell surface proteins besides EGFR with the same result. For instance, in other embodiments, the second antigen-binding domain may bind to low density lipoprotein receptor (LDLR), transferrin receptor (TfR), hepatocyte growth factor receptor (cMet), MHC Class II, vascular endothelial growth factor receptor (VEGFR) or other growth factor receptor, CD20, CD40, CTLA-4, OX-40, 4-1-BB or ICOS.

Moreover, it is to be understood that the orientation of the two antigen-binding domains is not fixed. The above embodiments can be modified such that the designated first antigen-binding domain binds to the cell surface protein, such as EGFR, and the designated second antigen-binding domain binds to the extracellular molecule, such as HIV.

In certain preferred embodiments relating to targeting HIV for degradation, the bispecific antigen-binding polypeptide is a bispecific VHH antibody.

As used herein, a method of “preventing” a disease or condition, means preventing the onset of the disease, preventing the worsening of symptoms, preventing the progression of the disease or condition or reducing the risk of a subject developing the disease or condition. As used herein, a method of “treating” a disease or condition means curing a disease or condition and/or alleviating or eradicating the symptoms associated with the disease or condition such that the patient's suffering is reduced. In the case of HIV, a method of preventing HIV may mean preventing or reducing HIV infection, preventing or reducing HIV entry into target cells, preventing or reducing HIV replication, preventing the onset of AIDS, preventing the worsening of AIDS-related symptoms, preventing progression of AIDS, or reducing the risk of a subject developing AIDS. A method of treating HIV may mean alleviating or eradicating HIV infection, or alleviating or eradicating the symptoms associated with AIDS.

### F. Targeting Sars-Cov-2 for Degradation

Another relevant target is Sars-Cov2. Neutralising antibodies have been generated in an attempt to block Sars-Cov2 infection. The present invention, utilising a targeted degradation approach, has the advantage that blocking of all envelope spike proteins is not necessary since binding of only a few envelope spike proteins should be sufficient to rapidly clear and degrade the virus. This will require a lower concentration of antibodies than neutralisation of all spike proteins. This allows for a mixture of different antibodies to be used, each targeting different epitopes, which may enhance effectivity and may decrease the chances of mutational escape.

As described in the below examples, the inventors have shown that the bispecific antigen-binding polypeptides of the invention can be used for the targeted removal and degradation of Sars-Cov2. The present invention thus also provides methods of treating or preventing Sars-Cov2. In this scenario, bispecific antigen-binding polypeptides may be engineered in which anti-Sars-Cov2 VHH is fused to anti-EGFR VHH such that Sars-Cov2 virus is internalised and degraded in EGFR-expressing fibroblasts and epithelial cells. It should be noted that much of the damaging effects of COVID-19 is due to secondary immunological responses of the body. The binding of antibodies to the viruses elicit a cascade of immunological reactions that in a percentage of cases has detrimental effects. The present invention on the other hand results in rapid clearance and degradation of virus and antibodies without further immunological activation being required (through the Fc tail that is present on natural antibodies), which will potentially circumvent these problems. Moreover, bispecific VHH single domain antibodies can be delivered intravenously and also by nebulized delivery (due their small size). Delivery of inhaled treatments to the respiratory system will result in lower dose requirements and direct engagement of the virus at the site of early infection. It is likely that bispecific VHH-mediated direct clearance of the virus by epithelial cells will be much more efficient than immunological clearance of virus that is just coated with neutralizing VHHs.

Accordingly, in certain embodiments, the bispecific antigen-binding polypeptide comprises:
a) a first antigen-binding domain that binds to Sars-Cov2; and
b) a second antigen-binding domain that binds to a cell surface protein.

In certain embodiments, the bispecific antigen-binding polypeptide comprises:
a) a first antigen-binding domain that binds to Sars-Cov2; and
b) a second antigen-binding domain that binds to EGFR.

In certain embodiments, the bispecific antigen-binding polypeptide comprises:
a) a first antigen-binding domain that binds to Sars-Cov2; and
b) a second antigen-binding domain that binds to a cell surface protein on a non-immunological cell, a fibroblast cell, epithelial cell, endothelial cell, or platelet.

In certain embodiments, the bispecific antigen-binding polypeptide comprises:
a) a first antigen-binding domain that binds to Sars-Cov2; and
b) a second antigen-binding domain that binds to EGFR on a non-immunological cell, a fibroblast cell, epithelial cell, endothelial cell, or platelet.

In each of the above embodiments, it is preferable that the extracellular molecule is internalised and degraded when both the extracellular molecule and the cell surface protein are bound to the bispecific antigen-binding polypeptide.

In each of the above embodiments, it is preferable that the extracellular molecule is degraded via the lysosomal pathway.

The present invention also provides a method of targeting Sars-Cov2 for cellular internalisation and degradation, the method comprising administration of a bispecific antigen-binding polypeptide or a pharmaceutical composition comprising same to a subject in need thereof.

The present invention further provides a method of treating or preventing Sars-Cov2, the method comprising administration of a bispecific antigen-binding polypeptide or a pharmaceutical composition comprising same to a subject in need thereof.

The present invention further provides, a bispecific antigen-binding polypeptide for use in a method of treating or preventing Sars-Cov2, the method comprising administration of a bispecific antigen-binding polypeptide or a pharmaceutical composition comprising same to a subject in need thereof.

The present invention further provides the use of a bispecific antigen-binding polypeptide for targeting Sars-Cov2 for cellular internalisation and degradation.

In each of the methods or uses described above, the bispecific antigen-binding polypeptide may be any of the bispecific antigen-binding polypeptides disclosed herein that bind to Sars-Cov2. In particularly preferred embodiments of the methods, the bispecific antigen-binding polypeptide comprises a) a first antigen-binding domain that binds to Sars-Cov2; and b) a second antigen-binding domain that binds to EGFR.

In each of the above embodiments in relation to targeting Sars-Cov2 for degradation, the skilled person is aware that the first antigen-binding domain may be engineered to bind to any epitope present on the surface of Sars-Cov2. For instance, in certain embodiments, the first antigen-binding domain may bind to a spike protein on the surface of Sars-Cov2. Similarly, the skilled person is aware that the second antigen-binding domain may be engineered to bind to other cell surface proteins besides EGFR with the same result. For instance, in other embodiments, the second antigen-binding domain may bind to low density lipoprotein receptor (LDLR), transferrin receptor (TfR), hepatocyte growth factor receptor (cMet), MHC Class II, vascular endothelial growth factor receptor (VEGFR) or other growth factor receptor, CD20, CD40, CTLA-4, OX-40, 4-1-BB or ICOS.

Moreover, it is to be understood that the orientation of the two antigen-binding domains is not fixed. The above embodiments can be modified such that the designated first antigen-binding domain binds to the cell surface protein, such as EGFR, and the designated second antigen-binding domain binds to the extracellular molecule, such as Sars-Cov2.

In certain preferred embodiments relating to targeting Sars-Cov2 for degradation, the bispecific antigen-binding polypeptide is a bispecific VHH antibody.

In the case of Sars-Cov2, a method of preventing Sars-Cov2 may mean preventing or reducing Sars-Cov2 infection, preventing or reducing Sars-Cov2 entry into target cells, preventing or reducing Sars-Cov2 replication, preventing the development of COVID-19, preventing the worsening of COVID-19-related symptoms, preventing progression of COVID-19, reducing the risk of a subject developing COVID-19, preventing the development or progression of post-COVID syndrome (long covid), preventing the worsening of post-COVID syndrome-related symptoms or reducing the risk of a subject developing post-COVID syndrome. A method of treating Sars-Cov2 may mean alleviating or eradicating Sars-Cov2 infection, or alleviating or eradicating the symptoms associated with COVID-19.

### G. Other Therapeutic Uses

Although the invention has been demonstrated for the targeted removal of HIV and SARS-CoV-2, it is clear that the concept can be applied for the removal of a broad range of extracellular molecules and thus to treat or prevent various diseases and disorders.

Accordingly, the present invention provides a method of targeting an extracellular molecule for cellular internalisation and degradation, the method comprising administration of a bispecific antigen-binding polypeptide or a pharmaceutical composition comprising same to a subject in need thereof.

The present invention further provides the use of a bispecific antigen-binding polypeptide for targeting an extracellular molecule for cellular internalisation and degradation.

In certain embodiments, the method or use comprises administering a combination of bispecific antigen-binding polypeptides, wherein the combination of bispecific antigen-binding polypeptides binds to more than one extracellular molecule, and/or more than one epitope on an extracellular molecule.

In certain embodiments, the bispecific antigen-binding polypeptide is administered orally, sublingually, topically, intravenously, subcutaneously, nasally, vaginally, rectally or by inhalation.

In certain embodiments, the degradation is via the lysosomal pathway.

The present invention further provides a method of treating or preventing an inflammatory pathology, optionally wherein the inflammatory pathology is selected from inflammatory bowel disease; psoriasis; rheumatologic inflammatory pathologies such as rheumatoid arthritis, ankylosing spondylitis; multiple sclerosis; autoimmune pathologies such as systemic lupus, erythematosus neuromyelitis optica; asthma; and allergies.

Also provided is a bispecific antigen-binding polypeptide for use in a method of treating or preventing an inflammatory pathology, the method comprising administration of a bispecific antigen-binding polypeptide or a pharmaceutical composition comprising same to a subject in need thereof. In certain embodiments, the inflammatory pathology is selected from inflammatory bowel disease; psoriasis; rheumatologic inflammatory pathologies such as rheumatoid arthritis, ankylosing spondylitis; multiple sclerosis; autoimmune pathologies such as systemic lupus, erythematosus neuromyelitis optica; asthma; and allergies.

As disclosed elsewhere herein, the extracellular molecule may be a viral antigen, and thus the invention contemplates methods of treating or preventing diseases and disorders characterised by viral infection.

The inventors have shown that hepatitis B virus-like particles can be efficiently targeted to the EGFR on cells in vitro. Upon targeting to the cells the virus-like particles are internalised and degraded in the lysosomes. This demonstrates the versatility of the invention and confirms that different viruses can be tackled in this way. Accordingly, the present invention also provides methods of treating or preventing hepatitis B.

Influenza viruses cause annual epidemics and occasional pandemics of respiratory tract infections that produce a wide spectrum of clinical disease severity in humans. As for SARS-CoV-2, both viral and host factors determine the extent and severity of virus-induced lung damage. The host's response to viral infection is necessary for viral clearance but may be deleterious and contribute to severe disease phenotypes. Influenza harbors haemagglutinin (HA) and neuraminidase (NA) spike proteins. HA play a key role in binding to sialic acid in the cell membranes resulting in internalisation. NA activity is subsequently required for proteolytic processing of the HA spikes leading to release of the viruses into the cytoplasm of target cells. The present invention is intended to result in internalisation via a different route, leading to lysosomal degradation. The present invention thus also provides methods of treating or preventing influenza.

As disclosed elsewhere herein, the extracellular molecule may be a toxin, and thus the invention contemplates methods of treating or preventing diseases and disorders characterised by toxicity.

Bacterial toxins in the circulation are also amenable to treatment according to the invention. Staphylococcus aureus is a major human pathogen that produces an array of toxins. Among the secreted toxins, the Staphylococcal superantigens (SAgs) play an important role in the debilitation of the host. They exert superantigenic activity that results in the activation of a large population of T cells, releasing large amounts of inflammatory cytokines. This overstimulation of the immune system can eventually lead to a systemic life-threatening response known as Toxic Shock Syndrome (TSS). Besides the administration of antibiotics, present therapy is primarily supportive, with fluid resuscitation and vasopressor agents. As current therapies are not sufficiently effective, novel treatments are required. Since toxic shock syndrome toxin, TSST-1, is regarded as the major etiologic agent of TSS, the specific removal of TSST-1 from the blood could be beneficial for patients suffering from TSS. Indeed, there is some evidence to support that plasmapheresis in severe cases of sepsis can ameliorate the course of the syndrome. The inventors have carried out studies in vitro which show that TSST-1 induces upregulation of MHC class II molecules on endothelial cells. The TSST-1 itself bound to these MHC class II molecules and was rapidly endocytosed and degraded (unpublished results). The application of bispecific antibodies will likely achieve the same result but more rapidly and more complete. With bispecific antigen-binding polypeptides targeting TSST-1 to EGFR or to suitable receptors on endothelial cells, the toxin will be targeted for rapid degradation. This is anticipated to be rapid enough to prevent the toxic effects of TSST-1 in the body. The inventors have generated VHH antibodies against TSST-1 that can be combined with anti-EGFR antibodies in bispecific antibodies to clear TSST-1. The present invention thus also provides methods of treating or preventing Toxic Shock Syndrome.

Since their first development and use in the late 1800s by Calmette, conventional antivenoms remain the only specific treatments for envenomation. Most antivenoms are composed of either whole IgGs (150 kDa), F(ab′)2 antibody fragments (100 kDa) or, in some cases, Fab antibody fragments (50 kDa) from horses or sheep immunized with one (monospecific) or a mixture (polyspecific) of venoms. Intravenous administration of antivenom is generally efficacious in treating systemic envenomation; however, because of the rapid development of localised pathologies and the inability of antivenom antibodies to penetrate affected tissues, conventional antivenoms are generally ineffective in treating local effects on tissues near the snake bite, often resulting in permanent physical disability. Furthermore, conventional antivenoms often elicit life-threatening adverse reactions such as anaphylaxis or serum sickness in patients. Recently, through construction of a VHH library from a llama immunized with crude N. kaouthia (monocle cobra) venom high affinity VHHs were isolated that offer full protection (100% mice survival) against α-Cbtx (α-cobratoxin).

Bispecific VHHs may much more rapidly clear and degrade the toxins. The present invention thus also provides methods of treating or preventing envenomation.

As disclosed elsewhere herein, the extracellular molecule may be a cytokine, and thus the invention contemplates methods of treating or preventing diseases and disorders characterised by excessive cytokine production.

Hyper-induction of pro-inflammatory cytokines, also known as a cytokine storm or cytokine release syndrome (CRS), is one of the key aspects of the currently ongoing SARS-COV-2 pandemic. This has similarly been described for other major human coronavirus and influenza A subtypes (H5N1, SARS-COV, MERS-COV, and H7N9). A recent study of various analyzed viruses highlights a SARS-COV-2-specific dysregulation of the type-I interferon (IFN) response and its downstream cytokine signatures. Targeted degradation of type I IFN may thus have a beneficial effect in preventing a CRS in COVID-19 disease development. Bispecific VHHs clearing type-1 IFN can be administered in suitable doses. Many other diseases result in cytokine imbalance. Balances can be restored by administration of combinations of bispecific VHHs against cytokines that are too highly expressed. Since the biheads themselves are relatively rapidly cleared from the body through the kidneys, this allows quantitative regulation of clearing the cytokine. The present invention thus also provides methods of treating or preventing cytokine release syndrome.

### H. Pharmaceutical Compositions and Route of Administration

The invention includes pharmaceutical compositions, containing one or a combination of bispecific antigen-binding polypeptides as described herein, which may be formulated with one or more pharmaceutically acceptable carriers or excipients. Techniques for formulating antibodies for human therapeutic use are well known in the art and are reviewed, for example, in Wang et al., Journal of Pharmaceutical Sciences, Vol. 96, pp 1-26, 2007, the contents of which are incorporated herein in their entirety.

In certain embodiments, the composition comprises a combination of bispecific antigen-binding polypeptides that bind to more than one extracellular molecule, and/or more than one epitope on an extracellular molecule.

The pharmaceutical composition according to the invention may be administered alone or in combination with other treatments, either simultaneously or sequentially. For instance, in certain embodiments in which the pharmaceutical composition is for use in treating or preventing HIV, the composition may be administered in combination with antiretroviral therapy, broadly neutralising antibodies, or other suitable treatment.

Pharmaceutically acceptable excipients that may be used to formulate the compositions include, but are not limited to: ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

The bispecific antigen-binding polypeptides are typically formulated as pharmaceutical compositions and administered to a subject in a “therapeutically effective amount”. As used herein, the term “therapeutically effective amount” is intended to mean the quantity or dose of a bispecific antigen-binding polypeptide, that is sufficient to produce a therapeutic effect, for example, the quantity or dose required to affect internalisation and degradation of the unwanted extracellular molecule, and/or to eradicate or at least alleviate the symptoms associated with a disease or condition. An appropriate amount or dose can be determined by a physician, as appropriate. For example, the dose can be adjusted based on factors such as the size or weight of a subject to be treated, the age of the subject to be treated, the general physical condition of the subject to be treated, the condition to be treated, and the route of administration.

The experimental results described herein indicate that even low concentrations of bispecific antigen-binding polypeptides results in a sufficiently high percentage of HIV-1 particles being decorated with the bispecific VHHs and therefore recruited to cells that display EGFR on their surface. Binding to a single envelope protein of HIV-1 is sufficient to target the virus to cells for degradation. This is in contrast to current approaches aimed at antibodies blocking viral adhesion to viral target cells which require that antibodies occupy all of the 14 spike surface proteins to be effective. The present invention thus requires small amounts of antibodies compared to traditional blocking methods. Consequently, the present invention readily allows the use of a combination of e.g. five or more different bispecific antigen-binding polypeptides, each with their own clade specificity. This will dramatically limit escape possibilities for the viruses.

For clinical use, in certain embodiments, the bispecific antigen-binding polypeptide as described elsewhere herein is administered to a subject as one or more doses of about 0.05 mg/kg body weight to about 20 mg/kg body weight. In certain embodiments, the bispecific antigen-binding polypeptide as described elsewhere herein is administered to a subject in a dose of about 0.05 mg/kg body weight to about 10 mg/kg body weight. In certain embodiments, the bispecific antigen-binding polypeptide as described elsewhere herein is administered to a subject in a dose of about 0.1 mg/kg body weight to about 10 mg/kg body weight. In certain embodiments, the bispecific antigen-binding polypeptide as described elsewhere herein is administered to a subject in a dose of about 0.5 mg/kg body weight to about 10 mg/kg body weight. In certain embodiments, the bispecific antigen-binding polypeptide as described elsewhere herein is administered to a subject in a dose of about 0.05 mg/kg body weight to about 5 mg/kg body weight. In certain embodiments, the bispecific antigen-binding polypeptide as described elsewhere herein is administered to a subject in a dose of about 0.05 mg/kg body weight to about 2 mg/kg body weight.

In certain embodiments, the composition is formulated for administration to a subject via any suitable route of administration including but not limited to orally, sublingually, topically, intravenously, intramuscularly, intradermally, transderamally, intraperitoneally, subcutaneously, nasally, vaginally, rectally or by inhalation.

Given their small size and robust thermostability, bispecific VHH antibodies of the invention can be aerosolized for the direct treatment of respiratory diseases.

During sexual intercourse HIV-1 can be transmitted. In addition to the use of condoms to reduce transmission, microbicides have been developed that neutralize the virus. Thus, microbicides containing the bispecific antigen-binding polypeptides as described herein can be administered as ointments directly to the vagina or anus where they can release the bispecific antigen-binding molecule locally. Moreover, localised production by Lactobacilli can be employed: for certain microbicides lactic acid bacteria can be used to steadily produce the microbicides. Certain species of Lactobacillus are commensals of the vagina and these species have been modified to produce certain microbicides. Thus, Lactobacillus, a natural commensal in the healthy vaginal microbiome, can be used to express bispecific antigen-binding polypeptides against HIV-1 in a soluble or in a cell-wall-anchored form, for example in Lactobacillus rhamnosus DSM14870 (Pant, Neha et al; 2006; The Journal of Infectious Diseases; vol. 194; 11:1580-8. doi: 10.1086/508747).

The invention will now be further understood with reference to the following non-limiting examples.

### EXAMPLES

### Example 1: Directing HIV-1 for Degradation by Non-Target Cells Using Bi-Specific Single-Chain Llama Antibodies

### Rationale

Described herein is a highly efficient method to clear HIV from the circulation and target it for destruction, using bispecific antibodies. These bind with one head to HIV and with the other head target the virus into cells in which it cannot replicate but will be degraded instead. The concept is that HIV is captured and destroyed by non-specialized, non-immune cells in the body. Removal of viruses from the circulation may alone or in combination with cocktails of antiretroviral drugs completely control and restrict HIV. To realize this concept, a series of bihead constructs were generated in which four different anti-HIV antibodies were coupled to two anti-EGFR antibodies that bind with high affinity to the epidermal growth factor receptor (EGFR). Small single chain llama antibody fragments (about 15 kD in size) that are called VHHs or nanobodies were used. Bispecific bihead antibodies of these were generated by cloning. The anti-HIV VHHs were chosen on the basis of broad binding capacity for different clades of HIV, including clades A, B and C. The EGFR is widely expressed in the body on cells that do not express HIV receptors. Since HIV receptors CD4 and co-receptors CCR5 and CXCR4 are required for productive replication of HIV, re-targeting HIV to these non-immunological target cells does not lead to viral replication. The inventors have demonstrated in vitro that the bispecific VHHs can bind HIV-1 envelope glycoprotein and deliver it to EGFR-expressing cells upon which the virus antigen-antibody complex is internalised and degraded by the lysosomal degradation pathway. This approach may be used to actively clear the virus from the body of infected patients.

### Construction and Functionality of Bi-Specific Bi-Head VHHs

Anti-HIV-1 VHHs (small llama antibody fragments) have been generated previously, yielding a considerable number of VHHs with broad binding capacity, including binders with blocking/neutralization activity. In the present study we investigated a novel approach by construction of bispecific VHH antibodies that are able to direct degradation of HIV-1 by non-target cells. We employed previously generated VHHs binding to the ectodomain of EGFR, including VHHs that were internalised after binding to EGFR. We combined four different anti-HIV-1 VHHs (1F10, H3, 2H10, 2E7) indicated here as H2, H3, H4 and H5 with two different anti-EGFR VHHs (EgA1, EgB4) indicated as E1 and E2. The anti-HIV-1 VHHs are specific for different envelope glycoprotein epitopes and were chosen on the basis of broad binding capacity for different clades of HIV-1, including clades A, B and C. The E1, E2 VHHs bind two different epitopes on the EGFR ectodomain with high affinity. Table 1 shows the bispecific VHH domains used and summarizes their characteristics. The bispecific VHHs (biheads) were generated by PCR cloning and contain a flexible linker sequence of 10 amino acids [(G₄S)₂].

TABLE 1
VHH llama antibodies combined in bispecific VHHs with a sequence of [G4S]₂
in between the VHHs. H is anti-HIV-1, E is anti-EGFR and Hep is anti-hepatitis B VHH.
Bispecific VHHs were generated in various combinations. E.g. H2E2 denotes a bispecific
VHH antibody with N-terminal VHH 1F10 linked to C-terminal EGb4. E1 (EgA1) is an
antagonistic anti-EGFR VHH that blocks EGF binding, whereas E2 (EgB4) is a non-
antagonistic antibody that binds to Domain I of EGFR.. The anti-HIV antibodies H2 and H3
were raised against gp120 envelope protein, H4 against gp4140 and H5 against gp140
protein. Hep1 binds the hepatitis B virus (HBV) surface antigen (HBs). This HBs is
expressed in HBV virus like particles (ADW & ADY). Bispecific VHHs generated in this study
are H2E2, E2H2, H2E1, H3E1, H4E2, E2H4, H5E2, Hep1E1 and control biheads E2E1,
H2H2, H5H4.

| VHHs | | | | | bispecific | bihead VHHs | |
|---|---|---|---|---|---|---|---|
| annotation | anti- | target protein | code in literature | ref. | antibody | bispecific | control |
| H2 H3 H4 H5 | HIV HIV HIV HIV | gp120 gp120 gp41 gp140/αgp41 | 1F10 this study 2H10 2E7 | 35 40 37 | 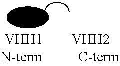 | H2E2 E2H2 H2E1 H3E1 | E2E1 H2H2 H5H4 |
| E1 | EGFR | EGFR domain III | EGa1 | 41 | | H4E2 | |
| E2 | EGFR | EGFR domain I | EGb4 | 41 | | E2H4 | |
| Hep1 | HBV | HBs in strain | S5 | 46 | | H5E2 | |
| | | ADW & ADY | | | | Hep1E1 | |

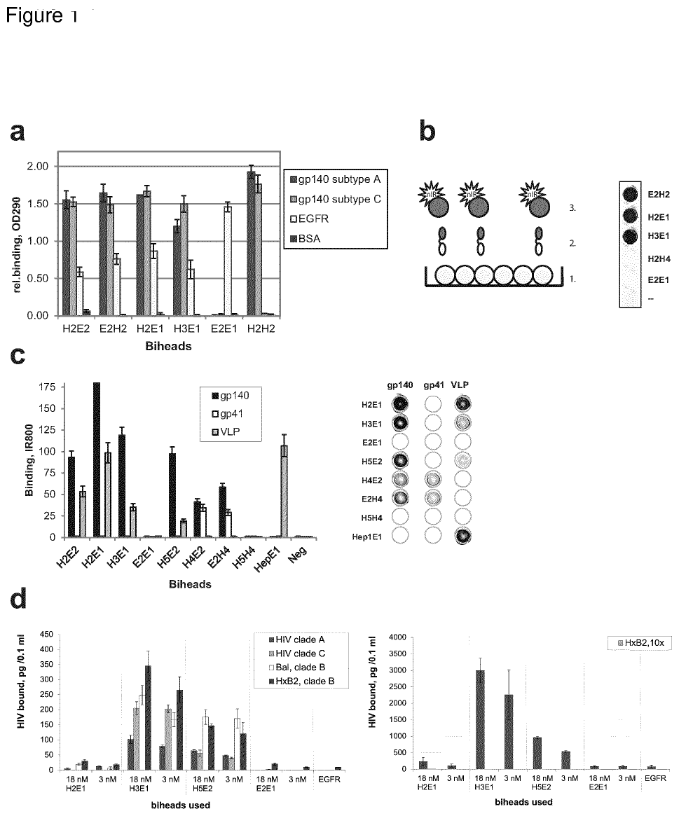

After fusion into a bihead, both the anti-HIV-1 and the anti-EGFR VHHs retain excellent binding properties, independent of the orientation of VHH antibody moieties in these molecules. This was demonstrated in ELISA analyses showing binding on purified EGFR ectodomain protein and on purified HIV-1 envelope proteins (clade A, 92UG037, and clade C, gp140ZM96) (FIG. 1a). Control proteins E2E1 and H2H2 demonstrated specificity of the VHHs as no binding was observed with these monospecific biheads to the other target. Next, we investigated whether the bispecific VHHs can recruit HIV-1 proteins to immobilized EGFR ectodomain. Instead of using multistep ELISA methods, we covalently labeled the HIV-1 proteins with the near infrared dye IR800CW, which allowed us to obtain quantitative binding results in IR800 FLISA (fluorescence linked immunosorbent assay). Using a scanner, bound IR800-labeled HIV-1 protein is directly detected in the wells. Clearly, unlike H2H4 and E2E1 controls, the bispecific VHHs mediate recruitment of the IR800-labeled envelope proteins to coated EGFR (FIG. 1b). We next expanded our panel of bispecific VHHs including VHHs binding to gp41. Functional envelope glycoproteins at the surface of viruses consist of trimers of covalently coupled gp41 and gp120 proteins, of which gp41 has a transmembrane region. In this study we used trimeric gp140 molecules that contain gp120 plus the ectodomain of gp41, stabilized by the addition of heterologous trimerization motifs at the C-terminus of the gp41 sequence. H2 and H3 bind to a gp120 epitope only and in accordance do not recognize gp41 trimers. H4 (α-gp41) binds the gp41 part of gp140 and H5 binds gp140³⁷ (FIG. 1c).

The recombinant glycoproteins gp41 and gp140 display a native conformation and form trimers. However, as the real HIV-1 virus particles are much larger and have complex membrane structures, we produced non-infectious HIV-1 virus-like particles (VLPs) that have the size of virions and express the ZM96 strain (clade C) envelope proteins in their membrane. In addition, to show that directed VHH-mediated recruitment is not limited to HIV-1 but may be more generally applicable, we also tested hepatitis B virus like particles using Hep, a VHH specific for this type of virus. The HIV- and hepatitis B VLPs were labelled with IR800 dye and are also shown to be specifically targeted to EGFR depending on the bispecific VHH used (FIG. 1c). Exceptions are H4E2 and E2H4 that target gp41 to EGFR, but not HIV-1 VLP, probably being too short for linking or due to other steric hindrance. Under the labeling conditions used, there were never any indications that labeling interfered with binding. In conclusion, we generated bispecific VHHs that can specifically target viral envelope glycoproteins as well as HIV-1 and HBV virus-like particles to EGFR.

Note that at modest to low antibody concentrations (nM) the VLP (pM) bind well to EGFR. The trimers contain three binding sites which will contribute to a higher avidity due to cooperative actions of multiple binding. This has been illustrated for gp140 trimers by quantitative FLISA experiments. Due to a large number of viral membrane proteins per particle, the VLPs contain even more binding sites, resulting in high avidity. Consequently, in competition experiments the preincubated VLPs, containing many binding groups at the surface of the particle were only 2-fold inhibited upon addition of 20-fold higher concentrations of competing free antibodies. It illustrates the power of binding avidity when multiple epitopes per particle are involved.

To test infectious HIV-1, two common HIV-1 reference strains HxB2 (clade B) and Bal (clade B) and two patient derived HIV-1 strains 92UG037 (clade A) and 96ZM651 (clade C), were cultured and tested for bispecific VHH mediated targeting to the EGFR ectodomain (FIG. 1d). Dependent on their binding efficiency for different clades the bispecific VHHs were shown to be effective and concentrations of 3 nM were sufficient to bind envelope proteins and target all four infectious HIV-1 strains to the immobilized EGFR. Since 3 nM of bispecific VHHs already provides a molar excess over HIV-1 envelope proteins, increasing the concentration to 18 nM hardly increased the amount of HIV-1 that is bound to EGFR. With a 10× increase in HIV-1 added, on average 8× more HIV-1 was targeted to EGFR (FIG. 1d), indicating the potency of the bispecific VHHs. In conclusion, bispecific VHHs can efficiently recruit HIV-1 reference strains and clinical isolates to EGFR ectodomain in vitro.

### Targeted Internalisation and Degradation

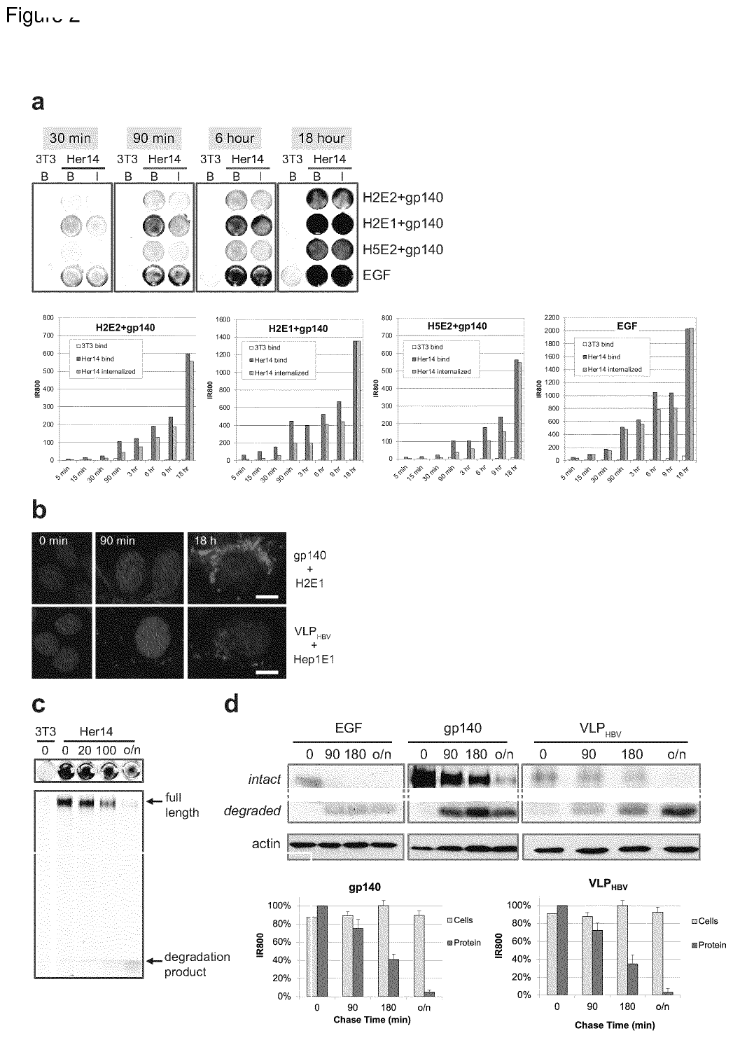

Next, we aimed to study bispecific VHH mediated binding of HIV-1 antigens to cells expressing EGFR, their internalisation and the intracellular fate of the internalised viral proteins. Viral antigens gp140 and VLPs were labelled with IR800 and pre-incubated with bispecific VHH for 30 mins. The resulting preformed antigen-antibody complexes were added to Her14 mouse fibroblasts cells expressing the EGFR receptor and to NIH3T3 2.2 cells that lack expression of EGFR. As a positive control for the experiment, we took EGF labelled with IR800. After binding for the indicated time points (FIG. 2a), cells were either washed to determine bound gp140-IR800 (panels B, bound) or they were acid stripped to determine internalised HIV envelope protein (panels I, internalised). As shown in FIG. 2a, we found that antigen-antibody complexes bound specifically to EGFR in Her14 mouse fibroblasts cells and not to negative control cells NIH3T3 2.2. Binding as well as the internalisation of HIV proteins increased continuously over 18 hrs. As expected, the positive control EGF-IR800 showed rapid internalisation (compare the differences between internalised and bound) as compared with the internalisation of HIV-1 envelope proteins. This is due to the fact that EGF-IR800 activates the EGFR receptor resulting in rapid internalisation. In contrast, our bispecific VHH antibodies do not activate the EGFR.

Nevertheless, we still observed continuous uptake of the bound envelope proteins increasing from 35%, 80%, to 95% after 90 min, 6 hrs and 18 hrs, respectively. We also found that Hepatitis B VLP (VLP_{HBV}) is a good model to investigate internalisation and fate of particles with the size of viruses, VLPs, in tissue culture. Gp140 and Hepatitis B VLPs were covalently labeled with AlexaFluor488 and internalisation was demonstrated by immune fluorescence microscopy (FIG. 2b). After 90 min incubation, labeled proteins were observed in endocytosed vesicles for both antigens. After overnight incubation all signal was present in endocytosed, mostly perinuclear, vesicles. Thus, we conclude that even without EGFR activation, bispecific VHH mediated targeting results in clear uptake of viral envelope proteins and VLPs by EGFR-expressing cells.

### Bispecific VHH-Mediated Uptake Results in Lysosomal Degradation

Binding of the natural ligand EGF to the receptor EGFR activates a negative feedback loop resulting in internalisation and degradation of receptor-growth factor complex. We assessed whether the bispecific VHH mediated uptake of the virus would also subsequently result in degradation of the complex components, even without EGF growth factor activation. Degradation was analyzed using covalently IR800 labeled EGF, gp140 and VLP_{HBV} proteins. Of note, we found that IR800 dye resides inside cells, even upon degradation of the carrier protein it is conjugated to. This allows for straightforward quantification of protein-linked and protein free dye in cell lysates. Cells were incubated with IR800-labeled proteins for various periods. After quantification of IR800 in the FLISA wells, we solubilized the cells and analyzed the quantity and intactness of the proteins by denaturing protein gel analyses, using quantitative analysis of IR800. FIG. 2c shows that upon internalisation of recruited HIV-1 envelope proteins (as in FIG. 2a), they become rapidly degraded (decrease of the full length gp140 signal). At 100 min of chase most dye is still present in (or on) the cells, however over 40% of the (internalised) envelope proteins are degraded and some IR800 labeled degradation products are visible on the gel. Similar results were obtained with the other bispecific VHHs. The degradation kinetics of recruited gp140 were compared with that of VLPs and the EGFR ligand EGF (FIG. 2d). As expected EGF is degraded more rapidly, degradation of both the HIV-1 proteins and VLPs is slower. However degradation was almost complete within a day. Western blot analyses measuring actin confirms that equal amounts of cell lysates were loaded in these experiments. Binding and degradation were similar when tested in other EGFR expressing cell types, including Hela, A431, and 14C cells.

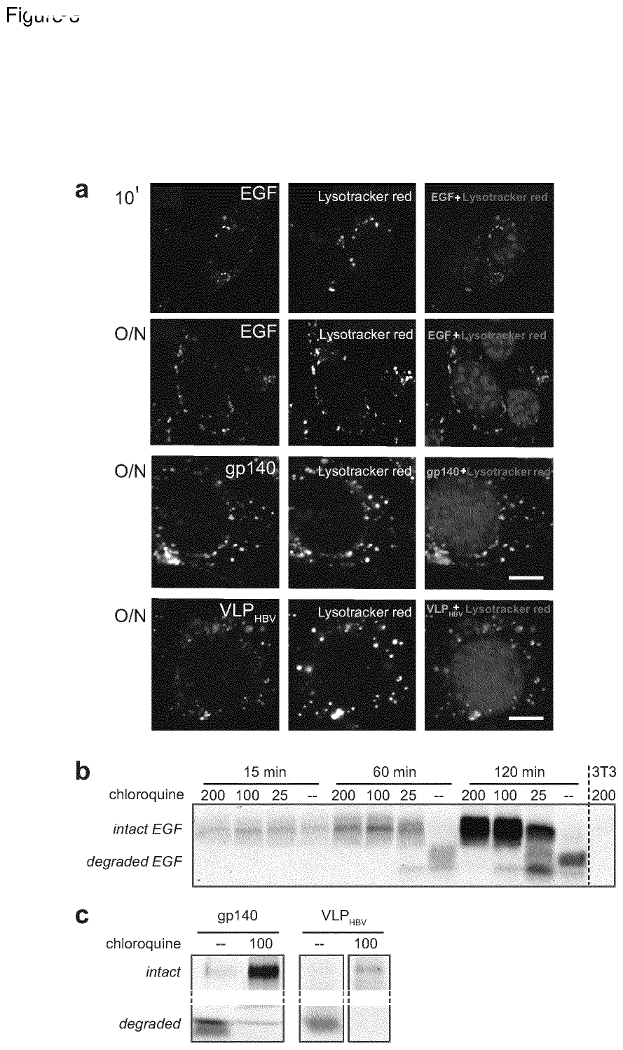

Next, we investigated the observed bispecific VHH mediated degradation of viral proteins in the EGFR-expressing cells. Upon EGF binding to the EGFR, the complex is internalised, sorted to early endosomes, late endosomes and finally degraded in the lysosomal compartment. Therefore, mouse fibroblasts cells Her14 were labelled with lysotracker dye that stains acidic compartments like lysosomes. After internalisation of labeled proteins (fluorescently labelled EGF, gp140 and VLP) mediated by bispecific VHH, the co-localization of the protein and lysotracker was analyzed with fluorescently labelled EGF, gp140 and VLP. Whereas FIG. 2a showed that at 30 min incubation most of the bound EGF is internalised, FIG. 3a shows that it is present in small endocytotic vesicles without co-localization with lysosomes. After 30 min co-localization occurs increasingly and it is completed in 3 hours (data not shown), which is maintained at least up to 18 hours (FIG. 3a, overnight panel [O/N]). After overnight incubation, fluorescent (degradation) products of EGF, recruited HIV-1 envelope proteins and VLPs show similar lysosomal localization. This result suggests that targeting mediated by bispecific anti-HIV-1 VHH for viral antigens to EGFR expressing cells leads to internalisation and degradation by the lysosomal degradation pathway

To further confirm these observations, mouse fibroblasts cells Her14 expressing EGFR receptor and negative control NIH3T3 2.2 cells were incubated with the lysosomal inhibitor chloroquine. The most appropriate functional range of concentration of chloroquine to inhibit lysosomal degradation in these cells was established with EGF-IR800, at chloroquine concentrations 0, 25, 100 and 200 UM (FIG. 3b). Chloroquine inhibits EGF degradation dose-dependently and is already optimal at 100 μM concentration. In cells not treated with chloroquine, internalised EGF is still intact after 15 min but completely degraded after 60 min. Similarly, FIG. 3c shows IR800 labeled envelope protein (gp140) and IR800 labeled hepatitis virus like particles (VLP_{HBV}), that were preincubated with the corresponding bispecific antibodies and incubated overnight on cells. Without chloroquine (-) they are largely degraded whereas 100 μM chloroquine prevented degradation, indicating that after uptake these proteins are targeted for lysosomal degradation. Further analysis of EGFR signaling pathways indicates that the internalisation does not per se require EGFR activation and is not accompanied by activation of signaling pathways associated with tumorigenic signaling.

### Bispecific VHH-Mediated Lysosomal Degradation of Infectious HIV by EGFR Expressing Cells

Next, the infectious HIV-1 reference strain HxB2 was analysed for targeted degradation in cells. Like many viruses HIV binds to heparin sulphate proteoglycans and thus also to the fibroblast cells. In vivo this “nonspecific binding” is thought to play a physiological role in infection by pre-concentrating the virion particles at the cell surface, as an intermediate step towards specific binding to CD4. To facilitate determining the specific effects of the biheads in our study, we firstly investigated methods for blocking non-specific binding using IR800 labelled HIV-VLP. None of these gave a satisfactory result, but bispecific VHHs containing an anti-HIV and an anti-EGFR moiety appeared to mediate on average 30% more binding than control biheads that contain two anti-HIV-1 moieties. Thus, for the VLPs the bispecific VHHs yield a modestly higher binding than background binding alone.

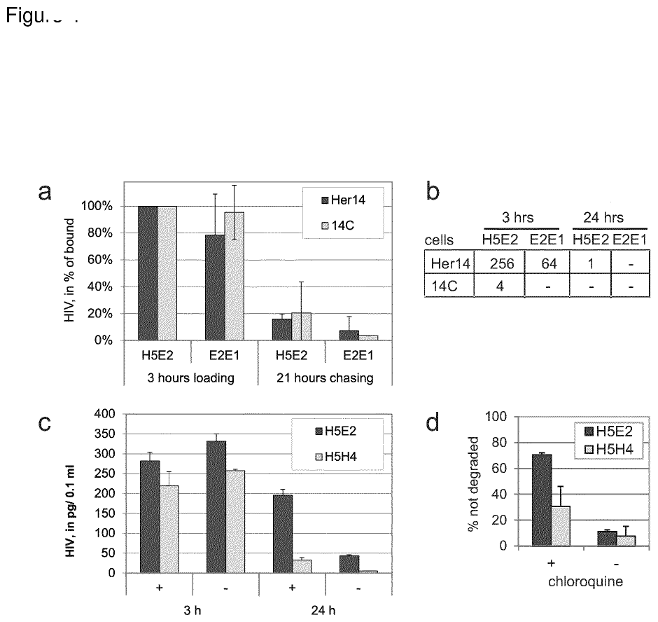

Subsequently, infectious HIV-1 HxB2 virus particles were incubated with bispecific VHH for 1 hour, to form HIV-antibody complexes. These complexes were then added to the wells containing either Her14 or 14C cells (both expressing EGFR), for 3 hrs (loading). Then, non-bound virus was removed by washing and the cells were left for 21 hours (chase). Viral protein was detected in lysates from treated cells using HIV-1 p24 ELISA. The amount of HIV-1 on or in the cells present after 3 hours binding (loading) decreased by 82-96% after 21 hours chasing (FIG. 4a), both with bispecific VHH antibody and negative control VHH antibody, due to release of the bound virus during the chase or due to internalisation and degradation. Whereas this was non-discriminative, trans-infectivity assays performed in parallel indicated that binding by bispecific VHH is 4 times more effective than controls in presenting HIV-1 to MT2 indicator cells (FIG. 4b). Virus was bound for three hours, washed and co-cultured with MT2 indicator cells for 7 days after which HIV-1 mediated syncytia were scored. This was performed with subsequent dilutions of the treated viruses and the highest dilution at which syncytia are observed is shown (FIG. 4b). Effectiveness of H5E2 was most clearly shown with Her14 cells. The 14C cells bound HIV as efficiently (FIG. 4a) but apparently present the virus in a less functional way for infection of the indicator cells to occur or the viruses are internalised so rapidly that they cannot infect the indicator cells anymore. After 21 hours chasing, infectious virus was no longer present at the surface of Her14 cells, suggesting that after initial loading/binding the virus is internalised and degraded. To test if degradation occurs in lysosomes, the cells were treated with chloroquine that inhibits lysosomal degradation whereas it does not influence internalisation (FIG. 4c). We show upon 3 hours binding with and without chloroquine and washing that HIV-1 is bound, with about 20% more binding with the H5E2 than with the control bihead H5H4. Subsequently, about 90% of the non-specifically bound virus (H5H4-treated) is lost during chasing for 21 hours, both in the presence and absence of chloroquine. With H5E2 in the presence of chloroquine 70% of the virus is still intact in the cells. This implies that despite a high level of nonspecific binding, 70% of the H5E2 treated viruses cells are internalised and degraded in lysosomes, as can be inhibited by chloroquine. Apparently, despite nonspecific binding the H5E2 targets the virus into the lysosomal degradation pathway. The graph in FIG. 4d indicates the percentage of HIV that was present after 24 hours as percentage of the input that is bound after 3 hours. Thus, most of the bound HIV-1 in the presence of bispecific VHH is mediated to lysosomal degradation that is inhibited by chloroquine. whereas the non-specifically bound HIV was released or, hypothetically, may follow a non-EGFR dependent internalisation and degradation route. In conclusion, the bispecific VHHs mediate targeting of infectious HIV to the lysosomal degradation pathway resulting in efficient degradation.

### CONCLUSION

In conclusion, we have generated many different bispecific antibodies to target HIV proteins to the EGFR. We tested HIV spike proteins, HIV virus-like particles and infectious HIV particles and show that all are efficiently targeted to EGFR. Also we investigated if the biheads are capable of targeting the HIV spike proteins to cells. Direct covalent labeling of HIV proteins and VLPs with near infrared dyes allowed rapid quantitative assessment. Her14 mouse fibroblast cells that express the human EGFR were compared with control NIH3T3 2.2 fibroblast cells that lack EGFR expression. In addition to binding also the level of internalisation was determined, by acid washing that removes proteins that are bound at the outside of the cell. It appeared that already at 3 hours incubation most bound spike proteins have been internalised into the cells and at 18 hours more than 95% has been internalised. Most importantly, using novel methods we demonstrate that at that time the internalised HIV proteins as well as VLP are almost completely degraded (>95%).

### Materials and Methods

### Construction of Bihead VHHs and Production

The anti-HIV VHHs 1F10 (H2), 2E7 (H5), 2H10 (H4), H3 (this study, sequence=EVOLVESGGGLVQPGGSLRLSCAASGSILDDANAMGWYRQTPGTERALVALITDSGATRY ADSVKG RFTISRDNAKNTATLQMNSLKPEDTAVYYCNFREFGGWGTNIDHWGQGTQVTVSS) and the anti-EGFR VHHs EGa1 (E1) and EGb4 (E2) (Hofman) were combined in biheads in various combinations using standard PCR techniques, with a linker encoding GGGGSGGGGS. The N-terminal VHHs were cloned using a 5′ primer containing a PstI digestion site (5′ GTTCCATTCTATGCGGCCCAGCCGGCC) and a 3′ primer encoding part of the 10 amino acid (AA) linker, including a BamHI digestion site (5′TCAGTAACCTGGATCCCCCGCCACCGCTGCCTCCACCGCCTGAGGAGACGGTGACC TG). The C-terminal VHH was amplified using a 5′ primer containing the second part of the linker including a BamHI digestion site (5′ AGGTTACTGAGGATCCGAGGTGCAGCTGGTGGAGTCTGG), while the 3′ primer contained a Not/digestion site (5′GGGACCCAGGTCACCGTCTCCTCA). PCR fragments were digested with PstI, BamHI and NotI (Fermentas), agarose gel purified, and cloned together into a phagemid vector for display on filamentous bacteriophage digested with PstI and NotI. Resultant clones contain a Myc-tag and a His tag. Expression was in E. coli TG1, DH10 or DH5α. Colonies were screened for insert by colony PCR using M13 primers. Expression of recombinant VHH proteins in E. coli and purification by immobilized metal ion affinity chromatography (IMAC) were performed with His tag binding Talon-beads (Clontech) as described³⁴. The isolated product checked for purity on a Coomassie-Blue-stained 15% SDS-polyacrylamid. All clones were confirmed by DNA sequencing.

Covalent Labeling of Proteins and VLP with Fluorescent Dyes

HIV envelope protein gp140 (UG37), subtype A, was provided. Recombinant HIV-1 envelope protein gp140CN54, subtype C, was obtained from the Centre for AIDS Reagents, NIBSC HPA UK, supported by the EC FP6/7 Europrise Network of Excellence, and NGIN consortia and the Bill and Melinda Gates GHRC-CAVD Project and was donated by Polymun, Immunodiagnostics, Immune Terchnology. gp41 (GCN) contains extracellular domains linked together to form a trimer. The HIV protein based VLP's (VLP_{HIV}) presenting the envelope protein ZM96 gp145, subtype C, were generated in 293T cells after large scale transient co-transfection with 2 plasmids encoding HXB2 Gag and ZM96 gp145 comprising the complete external gp120 moiety, the extracellular domain of gp41 as well as its transmembrane domain. VLPs harboring the HIV envelope proteins were sucrose gradient purified essentially as described. Hepatitis B virus like particles (recombinant surface antigen ADW subtype HC87-2) were obtained from HyTest Ltd. For labeling proteins concentrations were ≥1 mg/ml. If necessary, proteins were concentrated by Amicon® Ultra Centrifugal Filters or by Microcon YM-3 spin-columns (Millipore).

VHH and other proteins were labeled with a ratio of 20 μg:0.67 μg IRdye800 (IRdye 800CW NHS ester infrared dye from Licor, product 92970020) in PBS at room temperature, shaking, for half an hour. For the VLPs this was 20 μg with 2 μg dye and 1/10 vol 0.5M NaHCO₃ pH 9. Reactions were quenched with 10% of 2-(Methyl-Amino) ethanol (1M, pH 9.0, Sigma), and unbound dye was removed from the labeled protein by size exclusion separation over homemade 1 ml G-25 Sephadex (GE Healthcare) column. Protein concentration and labeling efficiency were determined with a Nanodrop 1000 spectrophotometer (Thermo scientific). For immune fluorescence proteins were labeled similarly with Alexa 488-NHS (Invitrogen).

### ELISA

96-well MaxiSorp plates were coated overnight with gp140 subtype A (=UG37), and subtype C (=CN54) or BSA control (250 ng/50 μl PBS). After blocking with 200 μl 4% w/v skimmed milk (Marvel, in PBS). VHHs were added for 1 hour (200 ng/100 μl). Incubations were in 1% Marvel in PBS, all washings with PBS with 0.05% Tween-20. Detection was with αMyc (Roche Diagnostics, 1/2000) or αHis (Amersham, 1/5000), peroxidase-conjugated secondary antibodies (Jackson Immunoresearch, 1:5,000) and o-Phenylenediamine (OPD). Secondary antibodies used were donkey anti-mouse and donkey anti-rabbit IgG.

### EGFR Binding of Biheads, FLISA and ELISA

Coating was with polyclonal Rabbit anti-Human IgG (DakoCytomation, 1/2000 in 50 μl PBS). After blocking with skimmed milk (4% w/v Marvel in PBS), incubation was with EGFR ectodomain (EGFR-ect) containing an Fc-tail (85 ng in 50 μl 2% Marvel) and next incubated with biheads at indicated concentrations in 1% Marvel. In FLISA results were analyzed by the Odyssey Infrared Imaging System (Li-Cor Biosciences). For ELISA αMyc (1/2000), αMouse-Ig-peroxidase (1/5000) and o-Phenylenediamine (OPD) were used.

### Preincubated Complexes

IR800 labeled proteins and VLP were preincubated with 2 to 3-fold molar excess biheads to form preincubated complexes for 30 min at room temp. (typically 50 ng gp140-IR800 or VLP-IR800+37 ng bihead VHH in 20 μl). For ELISA and FLISA preincubation was in 2% Marvel, for application on cells in 1% BSA (filter sterilized). Binding of preincubated complexes in assays was typically with 5 ng labeled protein in 200 μl i.e. 2 nM preincubated complex.

### Bihead Mediated HIV-1 Binding

The HIV-1 strains used were propagated and purified by standard procedures and resulting concentrations were determined by HIV-1 p24Ag ELISA (Aalto Bioreagents): HXB2, 940 ng/ml p24Ag; Bal, 109 ng/ml; 92UG037 (NIH Aids Reagent Program), 37 ng/ml; 96ZM651 (NIH Aids Reagent Program), 24 ng/ml p24Ag. Required amount of virus was spun for 1 h, 17000 rpm at 4° C. and the virus pellet was resuspended in PBS+2% BSA. 100 μl (typically 10 ng viral p24) was incubated with 60 ng or 10 ng bispecific bihead (resp. 18 nM and 3 nM bihead) for 1 h 37° C. for preincubation. Virus-bihead complexes were incubated in EGFR coated wells for 2 h. 37° C., washed 4 times with PBS and bound virus lysed in 100 μl 0.1% empigen (Sigma) in TBS which was transferred and analyzed by p24 ELISA

### Cell Culture

The murine fibroblast cell lines NIH 3T3 clone 2.2 (indicated 3T3) lacking EGFR expression and HER14 is derived from it as a stable transfectant expressing human EGFR. The tumor cell line UM-SCC-14C was kindly provided by G.A.M.S. van Dongen (Department of Otolaryngology, VU University Medical Center, Amsterdam, The Netherlands). Cells were cultured in Dulbecco's modified Eagle's medium (DMEM) supplemented with 7.5% fetal bovine serum (v/v), 100 U/ml penicillin, 100 μg/ml streptomycin and 2 mM L-glutamine (all Gibco, Invitrogen) at 37° C. in a 5% CO₂ humidified atmosphere. For incubations outside the incubator the medium was replaced by CO₂ independent medium (Gibco, Invitrogen)

### Internalisation Assays

Corning plate wells were coated with 0.25% gelatin (Merck, autoclaved) and washed with PBS. Her14 and 3T3 2.2 cells were seeded 4×10⁴ cells/well in a 48 wells plate and grown overnight in DMEM medium at 37° C. in a CO₂ containing environment. EGF-IR800 (LiCor) or preincubated complexes were added to the medium and incubated for 30 min, 90 min, 6 hours or 18 hours. Cells were washed twice with CO₂ independent medium, cooled on ice and stripped once for 5 min with strip buffer (250 mM NaCl, 100 mM Glycine, pH 2.5). Cells were washed twice with CO₂ independent medium and IR800 measured by Odyssey.

### Fluorescence Microscopy

Cover slips (Menzel-Glazer) in a 24 wells plate were coated with 0.25% gelatin and washed with PBS. 2×10⁵ Her14 cells or 3T3 2.2 cells per well were seeded on the cover slips and grown overnight. Cells were incubated with Alexa488 labelled EGF (Life technologies, 8 nM), or homemade Alexa488 labelled VLP_{HBV} (200 ng) and gp140UG37 (2 nM) that were preincubated with biheads. After PBS washing cells were fixed with 4% Paraformaldehyde (PFA) (Sigma) in the dark for 30 min at RT. Subsequently, cells were washed and treated with 20 ng/ml 4′,6-diamidino-2-phenylindole (DAPI, Roche Diagnostics Corporation) and embedded in Mowiol (Sigma) with PPD on a microscope slide. For colocalization with lysotracker cells were incubated with EGF-Alexa488 for 10 minutes or with preincubated complexes of bispecific VHH H2E1 with gp140-Alexa488 and Hep1E1 with VLP_{HBV}-Alexa488 for 6 hours, after which cells were washed 3 times with PBS and grown in fresh DMEM (8% FBS). After o/n growth 90 min before washing and paraformaldehyde fixing lysotracker red (Life technologies, 75 nM), was added to the cells. Images were obtained using a Zeiss Axiovert 200M confocal microscope (Carl Zeiss Microscopy GmbH, Germany) equipped with a 63× water-immersion objective (NA 1.2)

### Degradation Assay

Wells were coated with 0.25% gelatin. Her14 and 3T3 2.2 cells were seeded 4×10⁴ cells/400 μl/well in a 48-wells plate and grown overnight. Cells were cooled on ice and medium was collected as conditioned medium and replaced by ice cold CO2-independent medium containing 0.5% BSA (Sigma) and 0.4% FBS (300 μl/well). After washing once, EGF-IR800, bihead VHH or preincubated complexes were added (usually in 50 μl to mix rapidly) and incubated on the cells for two hours on ice in the dark. Cells were washed 3 times with the CO2-independent medium, wells were scanned for IR800. From a separate plate with the “0 min·chase” wells the medium was aspirated and replaced with 20 μl of 2× Laemmli sample buffer and lysed cells transferred to a PCR micro-titer plate (and stored on ice). For the other wells medium was replaced by the conditioned medium. Cells were transferred to 37° C. and 5% CO2 environment for 0 min, 90 min, 180 min or overnight. At these time points the cells were washed once, scanned for IR800 by Odyssey and subsequently lysed by 16 μl of 2× Laemmli sample buffer and transferred to a PCR micro-titre plate. Collected samples were boiled for 4 min at 95° C. in a sealed PCR micro-titer plate to prevent evaporation. Lysate was separated on a SDS-PAGE gel of 15% for EGF, VHH and VLP_{ADW} and 10% for the gp140. Samples were Western blotted on a PVDF membrane. Actin was detected with mouse anti-actin (MP Biomedicals, 1/10000) and donkey anti mouse peroxidase (DAMPO, 1/5000) followed by ECL, standard procedures.

### Chloroquine Treatment

48-wells plates were coated with 0.25% gelatin and 6×10⁴ Her14 or 3T3 2.2 cells were seeded per well. Cells were incubated with indicated concentrations chloroquine for 60 min. 3T3 2.2 cells with 200 μM. Next, the cells were incubated with 13 nM EGF-IR800, for 15, 60 or 120 min. After washing cells were scanned with the Odyssey. Then medium was removed and cells were lysed with 2× Laemmli sample buffer, boiled and analyzed on a 15% SDS page gel. IR800 was detected by Odyssey. After 60 min 100 UM chloroquine pretreatment, treatment with preincubated complexes (of gp140-IR800 with H₂E1 and VLP_{ADW} with Hep1E1, respectively) was overnight.

### Assays HIV-1 Binding to Cells

For HIV-1 binding, cells were seeded as above. The HIV strains were pre-incubated with biheads as described above but with 30 ng virus+60 ng biheads/200 μl conditioned medium. Where indicated heparin (Sigma) was added to 40 μg/ml for the last 30 min. of preincubation. Next the mixtures were added to the wells and plates were centrifuged for 10 min. 3000 rpm. and were incubated 3 hours or 24 hours while shaking (to mix properly). Cells were washed 4× with PBS and cells lysed in 0.1% empigen in TBS and subjected to p24 Elisa. For the trans-infectivity assay after incubation for 3 or 24 hours cells were washed 4× with PBS and MT2 indicator cells (NIH Aids Reagent Program) were added 40000 cells/200 μl/well in RPMI1640+L-Glutamine (Lonza) supplemented with 10% fetal bovine serum (v/v) (Sigma), 10 μg/ml gentamycine (Life Technologies). 24 and 48 hours later cytopathic effect (CPE) was scored. For pulse chase experiments cells were loaded with virus for 3 hours and after washing conditioned medium was added for chasing for 24 hours (or in some experiments in DMEM medium with 4% FBS, with similar result). 100 μM chloroquine (Sigma) treatment of cells started 2 hours before adding virus and chloroquine kept was present during the 3 hours loading. Chloroquine did not need to be re-added during 24 hours chasing for inhibition of degradation (results not shown).

### Example 2: Targeted Degradation of SARS-COV-2 Spike Proteins by SARS-COV-2 Specific Bispecific Single Chain Llama Antibodies

In addition to the targeted degradation of HIV- and Hepatitis B proteins, the inventor also performed similar experiments for another target, SARS-Cov-2. The VHHs C1 and C2 bind SARS-COV-2 spike protein with an affinity of <1 nM, and are neutralizing respectively at 2.5 nM for C1 and at 5.9 nM for C2. Bispecific antibodies were generated with a third anti-EGFR VHH, 7D12, indicated by E3. SARS-COV-2 spike proteins were labelled with IRdye800. Labelled proteins and biheads were preincubated and incubated with Her14 cells expressing EGFR. Binding was assessed in the tissue culture wells quantifying IR800. At indicated time points the cells in the wells were solubilized and analysed by PAGE. All essentially as described above, including control experiments to confirm the reagents (not shown).

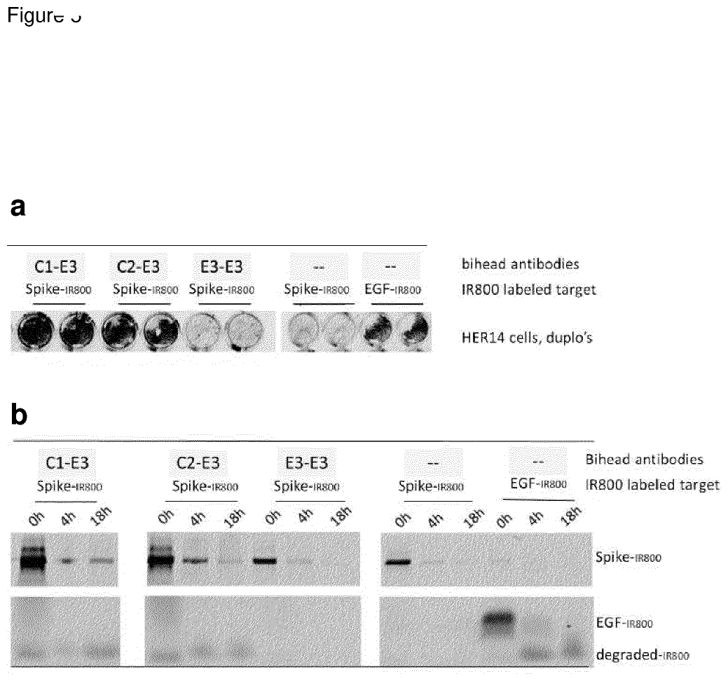

FIG. 5a shows that the bispecific biheads C1-E3 and C2-E3 specifically bind and target the spikeproteins to the EGFR expressing Her14 cells whereas the E3-E3 control bihead does not. A background amount of spike-IR800 protein binds also in the absence of biheads, yielding however a much lower signal. The PAGE gel analysis in FIG. 5b shows that the spike proteins targeted to the EGFR-expressing cells by C1-E3 and C2-E3 is largely degraded after 4 hours, similar to the EGF-IR800 control protein.

The results show that the invention can easily be applied to any extracellular target to which antibodies can be generated. Furthermore, it shows the versatility in use of antibodies that have different second antigen-binding domains and can therefore bind to different epitopes on a cell surface protein, i.e. EGFR (3 out of 3 were successful). No specific epitope is required for functionality with this invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

Various publications and patent applications are cited herein, the disclosures of which are incorporated by reference in their entireties.

1. A bispecific antigen-binding polypeptide comprising:
a) a first antigen-binding domain that binds to an extracellular molecule; and
b) a second antigen-binding domain that binds to a cell surface protein;
wherein the extracellular molecule is internalised and degraded via a lysosomal pathway when both the extracellular molecule and the cell surface protein are bound to the bispecific antigen-binding polypeptide.

2-3. (canceled)

4. The bispecific antigen-binding polypeptide of claim 1, wherein the bispecific antigen-binding polypeptide is a bispecific antibody, a bispecific antibody fragment, or a bispecific VHH antibody.

5. The bispecific antigen-binding polypeptide of claim 4, wherein the first antigen-binding domain is a VHH single domain, and the second antigen-binding domain is a VHH single domain.

6. The bispecific antigen-binding polypeptide of claim 1, wherein the extracellular molecule is a viral antigen, a toxin, a microbial pathogen, an allergen, a damaged or deregulated protein, an autoantibody, or other pathological or infectious agent.

7. The bispecific antigen-binding polypeptide of claim 6, wherein the extracellular molecule is a viral antigen, optionally selected from HIV, hepatitis, Sars-Cov2, influenza, herpes, Epstein Barr virus, adenovirus, flavivirus, echovirus, rhinovirus, coxsackie virus, respiratory syncytial virus, pandemic mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

8. The bispecific antigen-binding polypeptide of claim 7, wherein the extracellular molecule is a viral antigen selected from an HIV envelope glycoprotein and a Sars-Cov2 spike protein.

9. (canceled)

10. The bispecific antigen-binding polypeptide of claim 6, wherein the extracellular molecule is (i) a toxin selected from toxic shock syndrome toxin (TSST-1), snake toxin, cobratoxin (Cbtx), bacterial toxin, clostridium toxin, myeloperoxidase and an opioid; or (ii) a damaged or deregulated protein, such as a cytokine or growth factor, optionally selected from type-1 interferon (IFN), IL-6, PDL-1, GM-CSF, Gal-3BP, BAG3, IL-17 family, EGF, VEGF, NRG1, NRG2, NRG3, NRG4, HGF, RANK ligand, TNF-a, soluble TNF-a receptor, IL-Ib, IL-5, IL-17 A, IL-12, IL-23, C5, BAFF, IgE and TGFb.

11. (canceled)

12. The bispecific antigen-binding polypeptide of claim 1, wherein the cell surface protein is epidermal growth factor receptor (EGFR), low density lipoprotein receptor (LDLR), transferrin receptor (TfR), hepatocyte growth factor receptor (cMet), MHC Class II, vascular endothelial growth factor receptor (VEGFR) or other growth factor receptor, CD20, CD40, CTLA-4, OX-40, 4-1-BB or ICOS.

13-14. (canceled)

15. The bispecific antigen-binding polypeptide of claim 1, wherein the cell surface protein is present on a cell that is (a) capable of lysosomal degradation of the extracellular molecule, and (b) does not undergo lysosomal degradation of the extracellular molecule in the absence of the extracellular molecule and the cell surface protein being bound to the bispecific antigen-binding polypeptide.

16. The bispecific antigen-binding polypeptide of claim 15, wherein the cell is a fibroblast cell, epithelial cell, endothelial cell, blood cell or platelet.

17. The bispecific antigen-binding polypeptide of claim 1, wherein the first antigen binding domain binds to HIV and the second antigen-binding domain binds to EGFR.

18. The bispecific antigen-binding polypeptide of claim 1, wherein the first antigen binding domain binds to gp120, gp41 and/or gp140 on the surface of HIV.

19. The bispecific antigen-binding polypeptide of claim 1, wherein the first antigen binding domain binds to Sars-Cov2 and the second antigen-binding domain binds to EGFR.

20. A pharmaceutical composition comprising a bispecific antigen-binding polypeptide of claim 1.

21-22. (canceled)

23. A method of targeting an extracellular molecule for cellular internalisation and degradation, the method comprising administering a pharmaceutical composition of claim 20 to a subject in need thereof.

24. (canceled)

25. The method of claim 23, wherein the subject suffers from or is at risk of HIV infection and the pharmaceutical composition is administered for the treatment or prevention thereof.

26. (canceled)

27. A method of claim 23, wherein the subject suffers from or is at risk of Sars-Cov2 infection and the pharmaceutical composition is administered for the treatment or prevention thereof.

28. (canceled)

29. A method of claim 23, wherein the subject suffers from or is at risk of an inflammatory pathology selected from inflammatory bowel disease; psoriasis; rheumatologic inflammatory pathologies such as rheumatoid arthritis, ankylosing spondylitis; multiple sclerosis; autoimmune pathologies such as systemic lupus, erythematosus neuromyelitis optica; asthma; and allergies and the pharmaceutical composition is administered for the treatment thereof.

30-31. (canceled)

32. The method of claim 23, wherein the administration is orally, sublingually, topically, intravenously, intramuscularly, intradermally, transderamally, intraperitoneally, subcutaneously, nasally, vaginally, rectally or by inhalation.


SEQUENCE LISTING (6 sequences)

SEQ ID NO: 1 - protein, 10 aa, Synthetic construct
GGGGSGGGGS

SEQ ID NO: 2 - protein, 121 aa, Synthetic construct
EVQLVESGGGLVQPGGSLRLSCAASGSILDDANAMGWYRQTPGTERALVALITDSGATRYADSVKGRFTISRDNAKNTATLQMNSLKPEDTAVYYCNFREFGGWGTNIDHWGQGTQVTVSS

SEQ ID NO: 3 - DNA, 27 bp, Synthetic construct
  source 1..27: mol_type = other DNA
gttccattctatgcggcccagccggcc

SEQ ID NO: 4 - DNA, 58 bp, Synthetic construct
  source 1..58: mol_type = other DNA
tcagtaacctggatcccccgccaccgctgcctccaccgcctgaggagacggtgacctg

SEQ ID NO: 5 - DNA, 39 bp, Synthetic construct
  source 1..39: mol_type = other DNA
aggttactgaggatccgaggtgcagctggtggagtctgg

SEQ ID NO: 6 - DNA, 24 bp, Synthetic construct
  source 1..24: mol_type = other DNA
gggacccaggtcaccgtctcctca